(19) United States
(12) Patent Application Publication
(10) Pub. No.: US 2010/0286633 A1
(43) Pub. Date: Nov. 11, 2010

(54) Precision Lid Retracting Eyedropper Device

(76) Inventor: Alvin J. MARX, San Antonio, TX (US)

Correspondence Address: KAMMER BROWNING PLLC, 7700 BROADWAY, SUITE 202, SAN ANTONIO, TX, 78209, US

(21) Appl. No.: 12/722,501

(22) Filed: Mar. 11, 2010

Related U.S. Application Data
(63) Continuation-in-part of application No. 12/319,908, filed on Jan. 12, 2009.
(60) Provisional application No. 61/026,471, filed on Feb. 5, 2008.
(60) Provisional application No. 61/075,768, filed on Jun. 26, 2008.
(60) Provisional application No. 61/086,436, filed on Aug. 5, 2008.
(60) Provisional application No. 61/097,153, filed on Sep. 15, 2008.

(51) Int. Cl. A61F 9/00; A61M 35/00

(52) U.S. Cl. 604/296; 604/302; 604/298

(57) ABSTRACT

Precision lid retracting eyedropper device with a hollow, rigid housing having an upper aperture that allows a push button topped piston to enter the housing where the piston can impinge on a resilient tubular member that is part of a pump assembly. The housing also includes a solution storing chamber that feeds solution to the pump assembly. A solution exit tube attached to the outgoing portion of the pump assembly emanates from the bottom part of the housing and can be directed towards the user's eye. Integral downwardly facing resilient legs are placed just above and below the user's orbital ridge so that when the legs are squeezed and released, the user's eye lids are forced open allowing the user to dispense a precise amount of solution into his or her eye. A precision eye drop solution container having the ability to retain and dispense a precise single portion of eye drop solution each time the user depresses an actuator device located on and within the housing of the container. The container includes downwardly facing legs and feet that are parallel to each other and spaced so that the foot of each leg can align and make contact with the upper and lower orbital socket of the user. The feet have rubber-like under surfaces whose soft yet gripping quality acts to help hold the eye lids of the user in the open position. The dispensing action can be automated and an LED light indicating that dispensing has occurred.

### CROSS REFERENCE TO RELATED APPLICATIONS

This Application claims the benefit under Title 35 United States Code §120 as a Continuation-in-Part of co-pending U.S. patent application Ser. No. 12/319,908 filed Jan. 12, 2009, which itself further claims the benefit under Title 35 United States Code §119(e) of U.S. Provisional Application No. 61/026,471 filed Feb. 5, 2008; U.S. Provisional Application No. 61/075,768 filed Jun. 26, 2008, U.S. Provisional Application No. 61/086,436 filed Aug. 5, 2008, and U.S. Provisional Application No. 61/097,153 filed Sep. 15, 2008, the full disclosures of which are incorporated herein by reference.

### BACKGROUND OF THE INVENTION

1. Field of the Invention

This invention relates generally to the field of solution dispensing devices and more specifically to a precision lid retracting eyedropper device.

2. Description of the Related Art

Devices for dispensing eye drop solutions are well known. Generally, a bottle of eye drop solution includes a drop dispenser that is either built into the exit orifice of the container, or is a separate drop applicator comprised of a tubular main body having a tapered exit aperture on one end and a rubber squeeze bulb on the opposite end. In the first instance, to dispense the solution, the user squeezes that bottle forcing solution out of the exit orifice and into his or her eye. In the second instance, the user squeezes and releases the rubber bulb causing solution to be drawn up into the applicator. The rubber bulb is then squeezed to force the solution into his or her eye. Many users have trouble with both types of dispensing systems. The user has a tenancy to blink when the drop is about to enter the eye, causing the drop to miss the eye and land on a closed lid. Many of today's eye drop prescriptions call for precise dispensing of a specific amount of solution. Additionally, these prescription type solutions can be quite expensive, making any waste of solution quite costly. The precision needed for such applications can not be easily achieved with standard eyedropper devices. In addition, solution is wasted due to the user blinking during the application. Moreover, medication landing on the eyelid can cause skin irritation. Finally, eyedropper devices currently on the market do not have the ability to remain sterile after the first use. A clean and sterile eyedropper device would be preferable in many eye dropping applications.

A number of inventors have attempted to resolve the above mentioned problems. Thomas Keen, in his U.S. Pat. No. 4,543,096, discloses a dispenser with an eyelid opening device. The user is required to place a pair of lid spreading arms dangerously close to his eye and then to press a lever arm to keep the eyelids apart. No attempt is made to deliver a precise amount of solution. Thomas Sherman, in his U.S. Pat. No. 6,371,945, discloses an attachment for a bottle that includes a ring intended to help align the bottle with the eye. No attempt is made to hold the eyelid open or precisely meter the dose. Gary Campagna, in his U.S. Pat. No. 3,934,590, shows a tripod like device for aligning the solution bottle over the user's eye. No attempt is made to hold the lid open or to precisely measure the dose. James Dvaidian, in his U.S. Pat. No. 6,595,970, shows a device for dispensing eyed drops. He proposes a dispensing arm, one side of which includes an indentation that receives the user's nose, the other side of which accepts a dispensing bottle. The bottle includes a pair of arms which, when squeezed, impinge on the side walls of the bottle forcing solution out of the bottle and into the user's eye. No attempt is made to hold the user's eyelid open or precisely control the amount of fluid that exits from the bottle. The U.S. Pat. No. 4,692,834 of Julia Clifford et al shows a dispenser that facilitates the user's amount of drops that exit a solution holding bottle. This bottle has rotatable apertures that capture and release a drop of solution. James Walsh, in his U.S. Pat. No. 4,927,062, discloses a device that is intended to dispense a carefully measured amount of solution. However, no attempt is made to hold the device in the proper location or to hold the eyelid open. Manual Mosley's U.S. Pat. No. 5,578,020 shows a drop dispensing apparatus that attempts to control the amount of drops exiting from a bottle by pressing a deformable sleeve on the sides of the exit tube. No attempt is made to hold the eyelid open.

None of the above cited inventions dispenses a precise amount of eye drop solution and simultaneously holds the user's eyelids open while doing so. Additionally, none of the above cited inventions allows the eyedropper device to remain sterile after the first use.

### SUMMARY OF THE INVENTION

The primary object of the invention is to provide a precision lid retracting eyedropper device that includes a means to hold the user's eyelids in an open position during the application of eyed eye drops.

Another object of the invention is to provide a precision eyedropper device that allows the user to release a precise and consistent amount of eye solution by pressing an easy release button.

Another object of the invention is to provide a precision eyedropper device that can remain sterile during multiple uses.

Another object of the invention is to provide a precision eyedropper device that allows the user to remove and replace a cartridge containing eye drop solution from a hollow housing.

A further object of the invention is to provide an alternate embodiment that allows the user to remove and replace a cartridge containing individual doses within individual chambers that can be removed and replaced from an eye drop dispensing device.

A further object of the invention is to provide a precision eyedropper device that is easy and economical to manufacture.

Other objects and advantages of the present invention will become apparent from the following descriptions, taken in connection with the accompanying drawings, wherein, by way of illustration and example, an embodiment of the present invention is disclosed.

In accordance with the first embodiment of the invention, there is disclosed a precision lid retracting eyedropper device comprising: a hollow, rigid housing that encloses a solution storage bladder, a solution pump assembly, a solution expelling piston and an exit port. The upper portion of the piston exits an aperture located in the top portion of the housing and terminates in a push button. When the piston presses on the pump resilient assembly it causes a repeatedly consistent amount of solution to be dispelled from the exit port. The housing includes a pair of integral downwardly disposed resilient legs that can be squeezed, then placed above and below the user's orbital ridge and then released causing the user's eyelids to be spread and to remain in the open position during the eye drop dispensing event.

In accordance with a second embodiment of the invention, there is disclosed a precision lid retracting eyedropper device comprising: a hollow, rigid, elongate cartridge body having a top cover and a bottom tubular aperture, that dispenses liquid, said cartridge capable of retaining a liquid solution, a cartridge holding housing having a hollow portion capable of retaining said removable cartridge, said cartridge housing including an aperture to allow the said tubular aperture of said cartridge to protrude out of the bottom of said housing, said cartridge housing including a pair of integral downwardly disposed resilient legs, said cartridge side wall having an aperture that is covered by a resilient membrane, and said cartridge including a inwardly biased check valve located in said top cover portion and an outwardly biased check valve mounted within said cartridge in front of said bottom dispensing aperture.

In accordance with a third embodiment of the invention, there is disclosed an eyedropper device comprising: a main body and a removable and replaceable eye drop cartridge. The cartridge includes individual eye drop containing ampoules. The eye drop solution is ejected from the resilient ampoule when a spring biased piston presses down on the ampoule causing a precise amount of solution to be ejected out into the user's eye whose eyelids have been held open by downwardly disposed flexible legs.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is a perspective view of the first embodiment of the precision eye drop device of the present invention.

FIG. 2 is a front section view of the first embodiment of the eye drop device of the present invention with the storage cap in place.

FIG. 3 is a front section view of the first embodiment of the eye drop device with sterile tip and tip cover in place.

FIG. 4 is a front section view of the first embodiment of the eye drop device with a precise amount of eye drop solution exiting the device.

FIG. 5 is a top section view of the first embodiment of the eye drop device with the pump assembly in the ready position.

FIG. 6 is a top section view of the first embodiment of the eye drop device showing the resilient solution holding bladder.

FIG. 7 is a side view of the first embodiment of the eyedropper device of the present invention.

FIG. 8 is a front view of the first embodiment of the eyedropper device of the present invention.

FIG. 9 is a top view of the first embodiment of the eyedropper device of the present invention.

FIG. 10 is a bottom view of the first embodiment of the eyedropper device of the present invention.

FIG. 11 is a perspective view of a person using an alternate version of the first embodiment of the eyedropper of the present invention.

FIG. 12 is a perspective view of an alternate version of the first embodiment of the eyedropper of the present invention.

FIG. 13 is a front section view of an alternate version of the first embodiment of the eyedropper of the present invention in the ready position.

FIG. 14 is a front section view of an alternate version of the first embodiment of the eyedropper of the present invention in the use position.

FIG. 15 is a top section view of and alternate version of the first embodiment of the eyedropper of the present invention.

FIG. 16 is a side view of an alternate version of the first embodiment of the eyedropper of the present invention.

FIG. 17 is a front view of an alternate version of the first embodiment of the eyedropper of the present invention.

FIG. 18 is a top view of an alternate version of the first embodiment of the eyedropper of the present invention.

FIG. 19 is a bottom view of an alternate version of the first embodiment of the eyedropper of the present invention.

FIG. 20 is a perspective view of a second embodiment of the eyedropper of the present invention.

FIG. 21 is a front section view of a second embodiment of the eyedropper of the present invention in the ready position.

FIG. 22 is a front section view of the second embodiment of the eyedropper of the present invention in the use position.

FIG. 23 is a front view of the second embodiment of the eyedropper of the present invention.

FIG. 24 is an exploded view of the second embodiment showing the cartridge removed from the main housing.

FIG. 25 is a top view of the individual ampoules of the second embodiment of the eyedropper of the present invention.

FIG. 26 is a perspective view of a single ampoule of the second embodiment of the eyedropper of the present invention.

FIG. 27 is a side section view of the ampoule of the second embodiment of the eyedropper of the present invention in the ready position.

FIG. 28 is a side section view of an ampoule of the second embodiment of the eyedropper of the present invention in the use position.

FIG. 29 is a side view of the second embodiment of the eyedropper of the present invention.

FIG. 30 is a top view of the second embodiment of the eyedropper of the present invention.

FIG. 31 is a bottom view of the second embodiment of the eyedropper of the present invention.

FIG. 32 is a perspective view of a third embodiment of the eyedropper of the present invention.

FIG. 33 is a front section view of the third embodiment of the eyedropper of the present invention in the ready position.

FIG. 34 is a front section view of the third embodiment of the eyedropper of the present invention in the use position.

FIG. 35 is a front view of the third embodiment of the eyedropper of the present invention.

FIG. 36 is a top section view of the third embodiment of the eyedropper of the present invention.

FIG. 37 is a top view of the third embodiment of the eyedropper of the present invention.

FIG. 38 is a front perspective view of the third embodiment of the eyedropper of the present invention.

FIG. 39 is a partial section view of the third embodiment of the eyedropper of the present invention showing the trigger release assembly.

FIG. 40 is a perspective view of an alternate embodiment of the invention with folding legs in the folded down position.

FIG. 41 is a perspective view of an alternate embodiment of the invention with folding legs in the folded up position.

FIG. 42 is a partial section view of an embodiment of the exit orifice of the present invention that produces a spray effect.

FIG. 43 is a perspective view of the fourth preferred embodiment of the invention

FIG. 44 is a section view of the fourth preferred embodiment of the invention.

FIG. 45 is a front view of the fourth preferred embodiment of the invention packaged with a separate syringe containing eyed drop solution.

FIG. 46 is a front view of an air tight package with a syringe attached and ready to inject eye drop solution from the syringe into the bladder of the fourth preferred embodiment.

FIG. 47 is a front section view of an air tight package with a syringe attached the fourth embodiment and ready to inject eye drop solution from the syringe into the bladder in a sterile manner.

FIG. 48 is a partial section view of the needle insertion into the fourth preferred embodiment of the invention.

FIG. 49 is a perspective view of the fifth preferred alternate embodiment of the invention.

FIG. 50 is a front section view of the fifth preferred alternate embodiment of the invention.

FIG. 51 is a side section view of the fifth preferred alternate embodiment of the invention.

FIG. 52 is a partial section view of the portion pack strip of the fifth preferred alternate embodiment of the invention.

FIG. 53 is a partial section view of a single portion pack after it has been depressed by the plunger tip.

FIG. 54 is a front view of the portion pack strip.

FIG. 55 is a top view of the portion pack strip.

FIG. 56 is a front section view of the fifth preferred alternate embodiment of the invention with the portion pack strip removed.

FIG. 57 is a front view of the fifth preferred alternate embodiment of the invention.

FIG. 58 is a rear view of the fifth preferred alternate embodiment of the invention.

FIG. 59 is a perspective view of the fifth preferred alternate embodiment of the invention having the resilient leg design.

FIG. 60 is a front view of the fifth preferred alternate embodiment of the invention having the resilient leg design.

FIG. 61 is a perspective view of the fifth preferred alternate embodiment of the invention having the resilient leg design.

FIG. 62 is a front view of a sixth preferred embodiment of the precision lid retracting eyedropper device of the present invention.

FIG. 63 is a front section view of a sixth preferred embodiment of the precision lid retracting eyedropper device of the present invention.

FIG. 64 is a front section view of the sixth preferred embodiment of the precision lid retracting eyedropper device of the present invention.

FIG. 65 is an exploded view of the portion pack strip of the fifth preferred alternate embodiment of the invention.

FIG. 66 is a partial perspective view of the rear side of the portion pack strip.

FIG. 67 is a perspective view of an automated version of the fifth preferred alternate embodiment of the invention.

FIG. 68 is a front section view of the automated version of the fifth preferred alternate embodiment of the invention.

FIG. 69 is a perspective view of the portion pack strip showing a cap tether.

FIG. 70 is a flow chart schematic of the sequence of events when using the invention.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

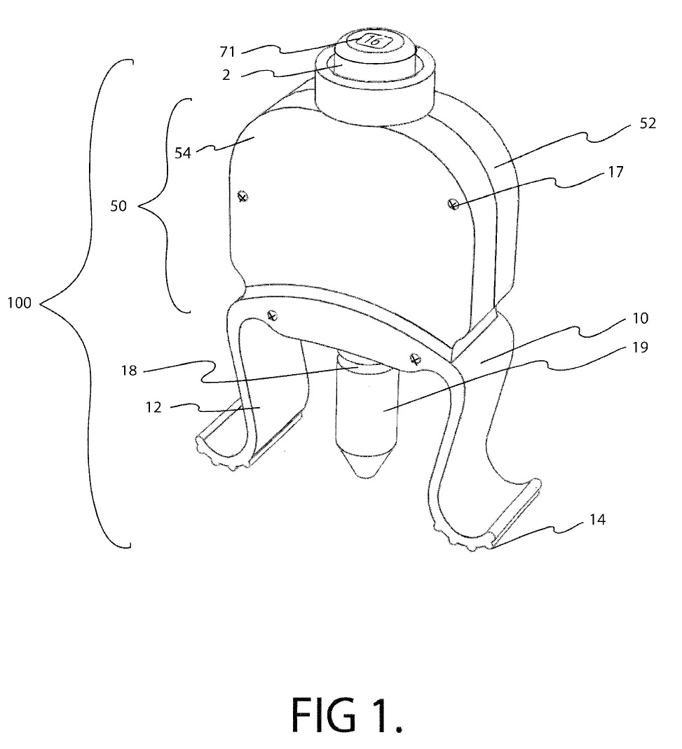

Referring now to FIG. 1 we see a front perspective view of the first embodiment of the precision eyedropper of the present invention 100. A hollow rigid housing 50 includes an integral resilient pair of downwardly facing legs 10, 12, that terminate in J shaped feet. Traction strips 14, located on the underside of each foot are designed to help stretch the user's orbital skin during operation as will be described below. The housing assembly 50 includes a back half 52 and a front half 54 held together by screws 17 or other standard fastening means.

Push button 2, when pushed, causes a precise amount of eye drop solution to be dispensed and will be described fully below. Push button 2 includes a standard counter 71 that advances one number after each use and will be described more fully below. Exit cover 19 protects the tip portion of the device 100 during storage. For ultimate sterility performance, a plurality of exit covers 19 can be included with the device 100 and a new cover 19 can be installed after each use. Inwardly facing detent 18 allows exit cover to be removable retained on the exit portion of the device 100.

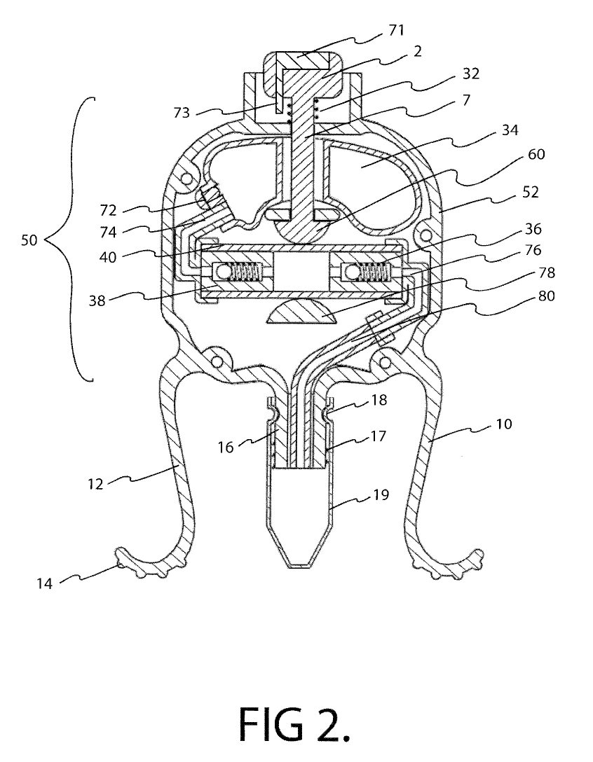

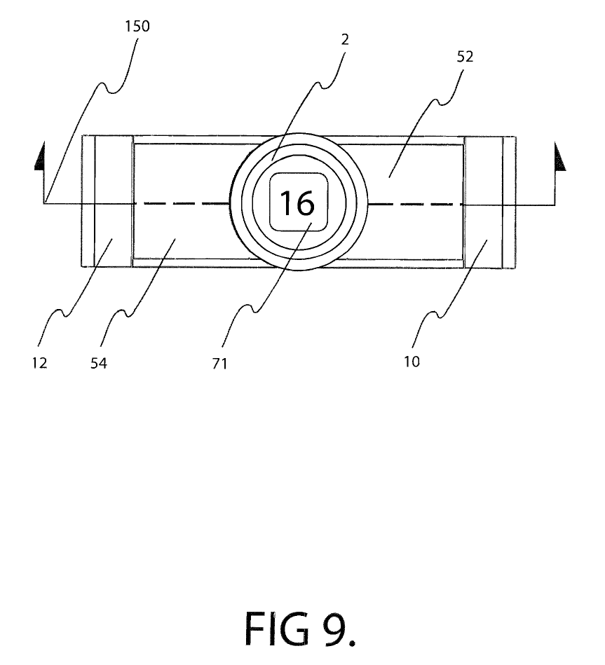

FIG. 2 shows a front section view of the invention 100 as defined by section line 150 shown in FIG. 9. Solution holding bladder 34 is located in the top portion of the housing 52. A pump assembly comprising an inwardly direct check valve 38, and an outwardly directed check valve 37 and resilient tube 40 is located in the central portion of the housing 52. A slidable piston 7 is spring biased 32 and terminates at its top portion in a push button 2 that includes a standard counting device 71. Each time the push button 2 is pushed down, tip 73 engages floor 75 which advances the counter one by one number. In this way, the user can keep track of how many dispensing events he has engaged in and therefore estimate how many dispensing events are left within the unit 100. The opposite end of piston 7 terminates in a pump actuator tip 60 that engages horizontally disposed resilient pump tube 40. Bottom tube retainer 78 holds tube 40 in place when the top of tube 40 is pushed down by piston tip 60. The pump tube 40 terminates in left cap 39 and right cap 41. A solution transfer tube 74 carries solution from bladder 34 to left intake check valve 38. Upper exit tube 76 carries solution from outgoing check valve 37 to lower exit tube 80 which is surrounded by rigid tube 16. Lower exit tube 80 terminates in rubber stopper 25. Cap 72 can be removed for filling bladder 34 with solution and then be replaced when bladder 34 is full.

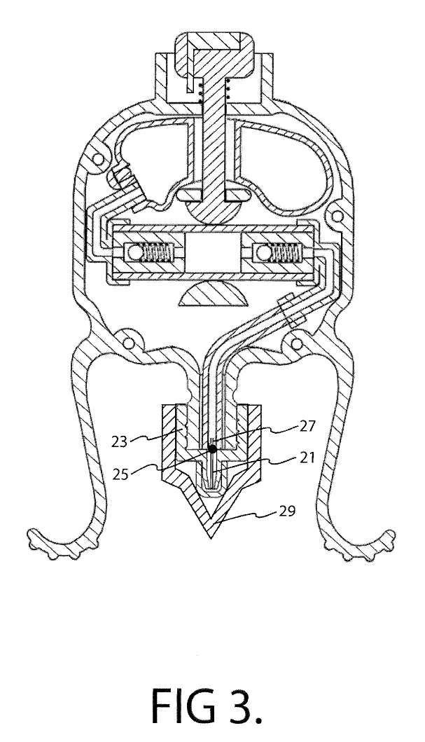

FIG. 3 shows the same front section view as in FIG. 2 with a sterile cap assembly comprised of outer cap 29, removable inner cap 27, and threaded cap 23 that includes final needle type exit tube 21. When the threaded 17 cap 23 is installed by the user, the top portion 27 of needle 21 pierces rubber stopper 25 located at the base of lower exit tube 80. The top portion of outer cap 29 is sealed with a peal off label, not shown, keeping the threaded cap 23 and attached needle 21 in a sterile condition until the time of use. The entire sterile cap assembly is removed after use and a new sterile cap assembly is installed at the time of the next use. This type of replaceable sterile cap system is known and accepted as proper sterile procedure. This system can currently be found in the product “Forteo” teriparatide injection, manufactured by Lily France SAS.

This system insures that this version of the eyedropper of the present invention remains sterile for each use. This form of tip that maintains sterility can be applied, if so desired, to any or all embodiments of the invention shown below.

FIG. 3 also shows an alternate embodiment of the location of the bladder fill cap 72A so that a person can fill or re-fill the bladder 34 without having to open the housing half 54.

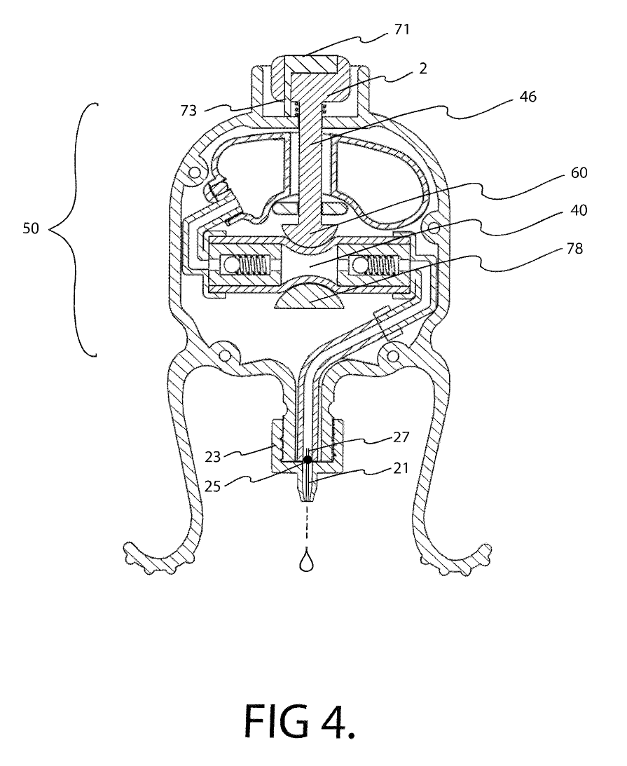

FIG. 4 shows a section view of the first embodiment after a person has pressed down on push button 2 causing deformation of resilient pump tube 40 forcing a precise amount of solution out of check valve 37 and out of exit tip 21 as shown by droplet 29. When the push button 2 is released by the user, the resulting suction caused by tube 40 returning to its original position forces a new amount of solution into incoming check valve 38. Because solution bladder 34 is made of resilient material such as latex rubber, the bladder 34 can collapse as the solution contained within the bladder diminishes. The wall of the resilient bladder 34 is approximately five thousandths of an inch thick so that it is extremely flexible and can completely compress as liquid is pumped out of the bladder, ultimately leaving the bladder completely devoid of liquid and of air. In this way, no air needs to enter the system thereby insuring that the dispensing of solution remains precise and air free.

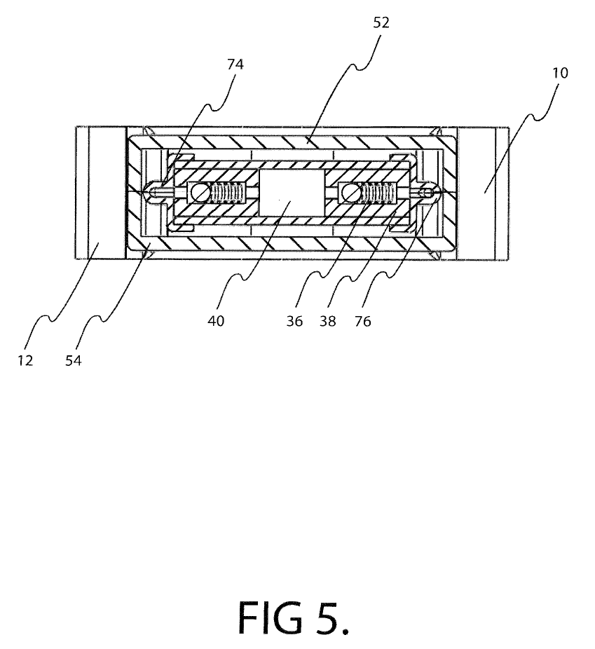

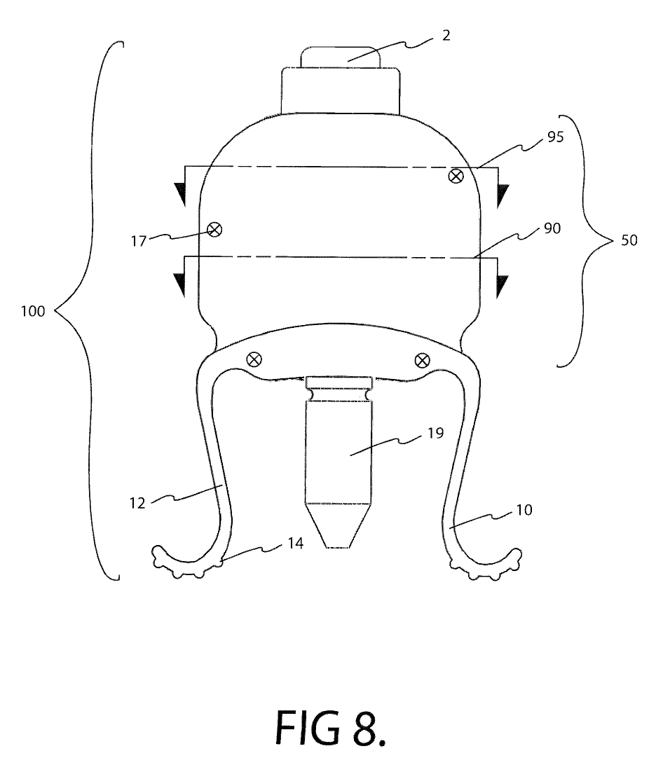

FIG. 5 shows a top section view as defined by section line 90 shown in FIG. 8. Tube 40 is clearly shown to be enclosed within housing halves 52, 54. Identical Check valve bodies 37, 38 include standard compression spring 36 and valve ball 39. Valve entrance tube 74 can be seen as well as exit tube 76.

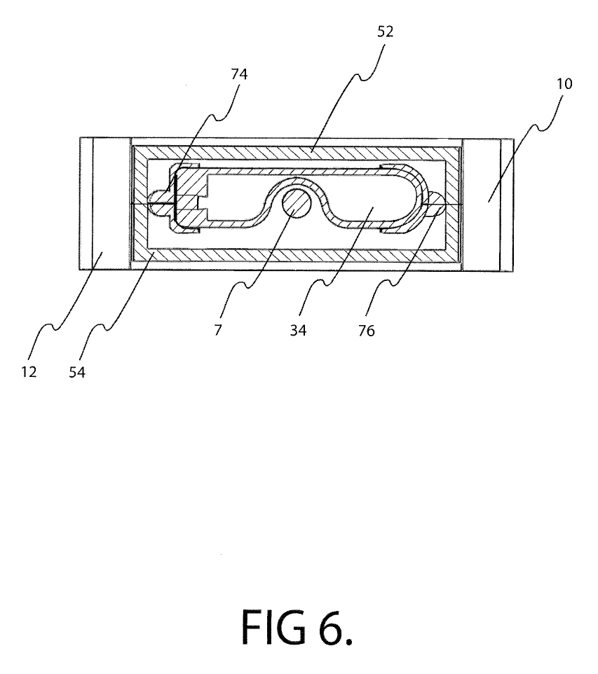

FIG. 6 shows another top section view as defined by section line 95 shown in FIG. 8. This view shows how bladder 34 deforms to accommodate piston 7.

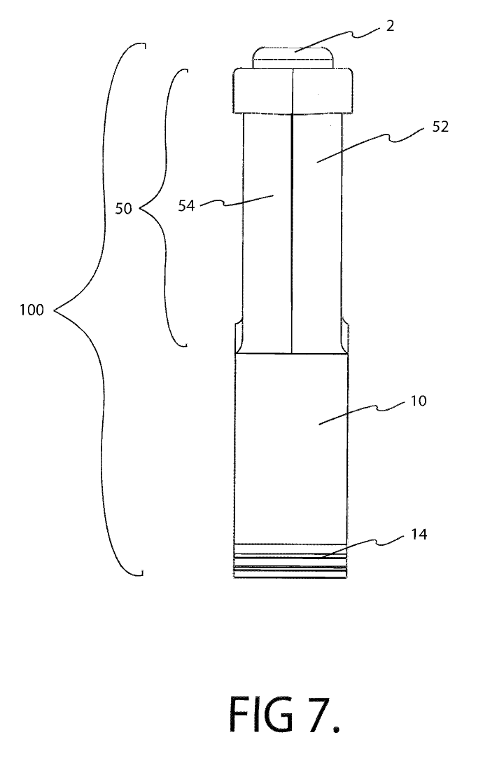

FIG. 7 shows a side view of the first embodiment of the invention 100. Housing halves 52, 54 can be clearly seen.

FIG. 8 shows a front view of the first embodiment of the invention 100 with storage cover 19 in place. The housing halves 52, 54 are held together by screws 17 or other standard fasteners. Resilient molded plastic legs 10, 12 can be clearly seen as well as traction tips 14 which are constructed of high friction material such as rubber.

FIG. 9 shows a top view of the first embodiment of the invention where counter numbers 71 can be clearly seen.

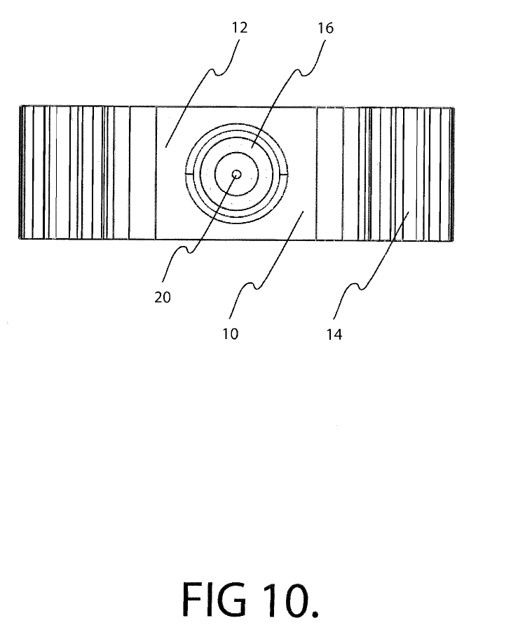

FIG. 10 shows a bottom view of the first embodiment of the invention where friction strips 14 can be clearly seen as well as needle tip 21 and rigid tube 16.

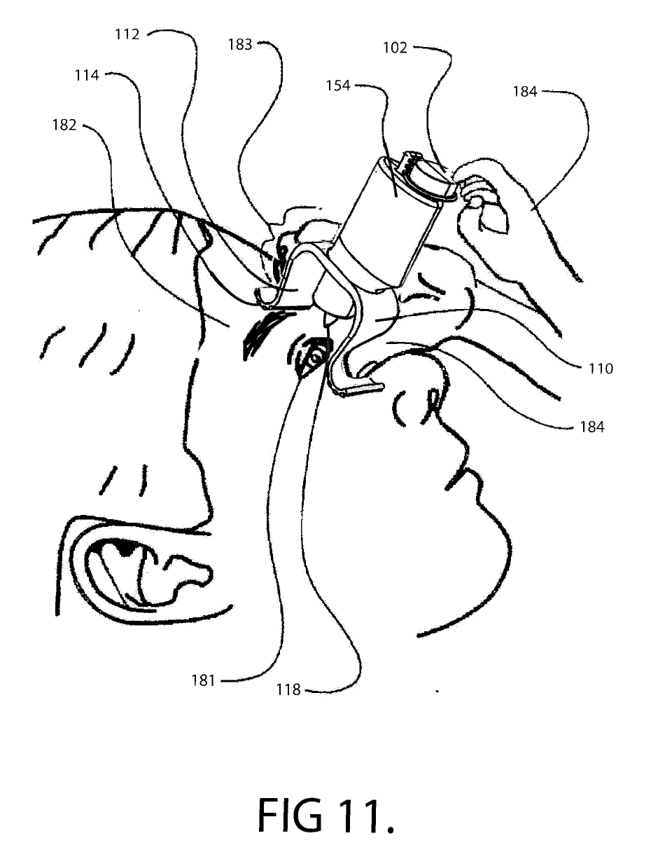

FIG. 11 shows a perspective view of a person 182 getting ready to administer a precise amount of solution to her eye 181 using a second variation 200 of the first embodiment of the invention. The user holds the legs 110, 112 with one hand 183, 184 as shown, then squeezes the legs, and then places the traction tips 114, 116 of the legs 410, 412 onto the lower and upper portions of the skin of the orbital ridge as shown. The skin overlying the orbital ridge is tougher and less likely to be damaged than eyelid skin. The user places the forefinger 184 of her other hand on activation button 102. The location of activation button 102 makes it easy for the user to operate the device with the use of only one finger. The user then releases legs 110, 112 causing the legs to spread outward and make traction tips 114, 116 spread the user's eyelids so that she cannot blink. The configuration and placement of the legs 110, 112 during use provides traction on the skin over the orbit region making it possible to hold the lids open with minimal discomfort to the user. The skin of the eyelids is much thinner than the skin over the orbit and would be more easily damaged if the legs were placed directly on the eyelids. The user has been instructed to gaze upward, thereby bringing the sclera of the eye directly under the fluid aperture. The sclera is less sensitive to irritation from medication than the cornea. After this has been achieved, the user presses on activation button 102 causing a precise amount of solution to quickly exit from aperture 118. With eyelids held apart by legs 110 and 112, the user is less likely to blink and therefore can be more certain that the act of administering eye drop solution will be successful. This leg configuration and lid spreading action is found in all embodiments of the invention shown above and below. The location of the solution exit tip 118 is in close proximity to the eye 181 so that the chance of missing the eye during solution application is minimized. The above described holding and dispensing position anchors and stabilizes the dispenser making it easier for the user to deliver the eye drop solution into his or her eye thereby conserving potentially expensive eye drop solution.

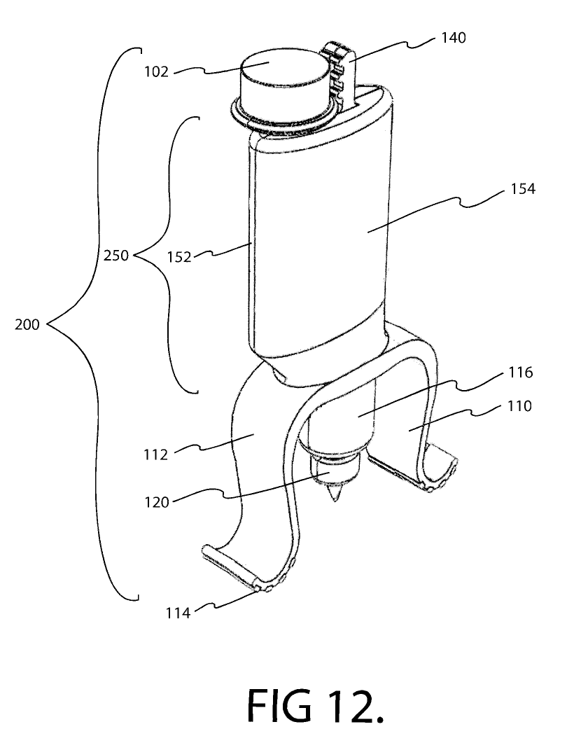

FIG. 12 shows perspective view of an alternative version of the first embodiment of the invention 200. This version works in a similar way as the original first version however its housing 250 has a more vertical orientation. This version 200 also shows a method of adjusting the amount of solution dispensed by the user as will be explained in detail below.

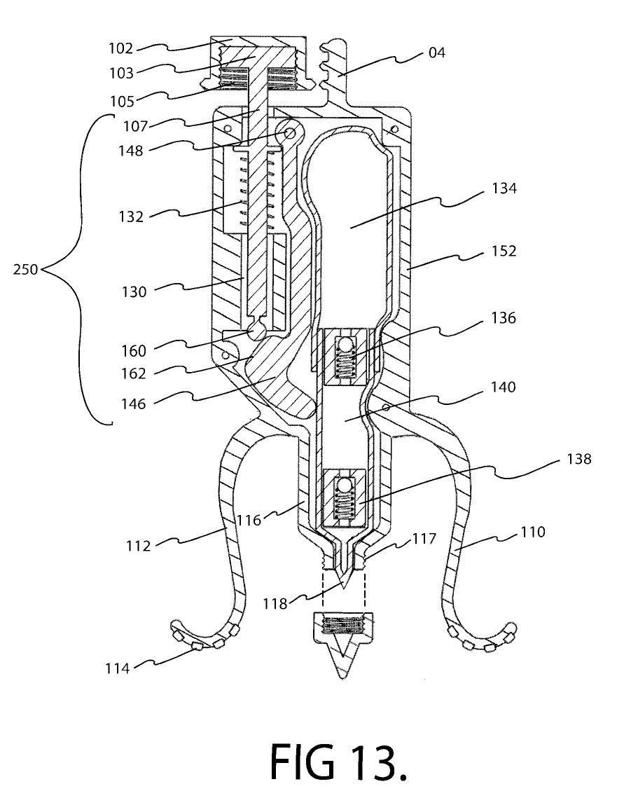

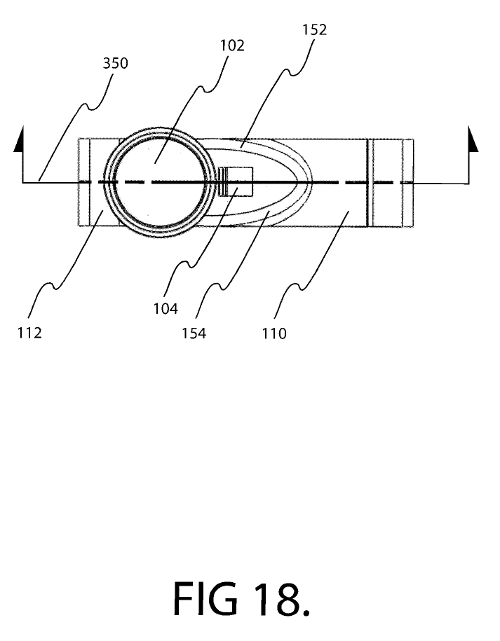

FIG. 13 shows a front section view of the alternate version 200 of the first embodiment of the invention as defined by section line 350 of FIG. 18. In this version, the contents of the housing 250 can be seen. The bladder 134 is situated in a vertical format and the pump tube 140 is also situated in a vertical position. This version allows a more direct dispensing of solution with no need for entrance and exit tubes as shown in the first version of the invention 100. In this version 200 the user presses push button 102 and piston 107 terminates in a ball member 160 which engages ramp 162 of pinned 148 push foot 146. This embodiment does not show the sterile tip described in the first embodiment, however it can be added if so desired. This version includes a threaded removable and replaceable cap 119 that can engage the mating threads 117 locate at the exit portion of tube 116. The check valve setup 138, is identical to that of the first version 100. In version 200 push button 102 includes an internal thread 105 that engages mating threads on the piston 107. When the user turns the knob clockwise, he or she reduces the amount of solution delivered when the push button is pressed because the distance the piston travels decreases the movement of the push foot 146 thereby decreasing the amount of incursion into resilient tube 140 and reducing the amount of solution that will be ejected from tip 118. Indicator notches on post 104 help the user determine the amount of solution to be dispensed.

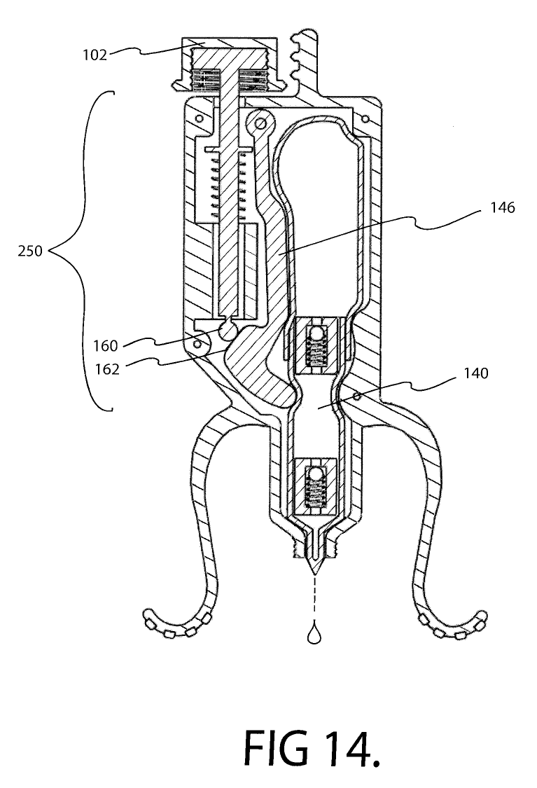

FIG. 14 shows a the alternate version of first embodiment 200 in the use position where tube 140 has been squeezed by push foot 146 causing a drop 129 of solution to exit from tip 118.

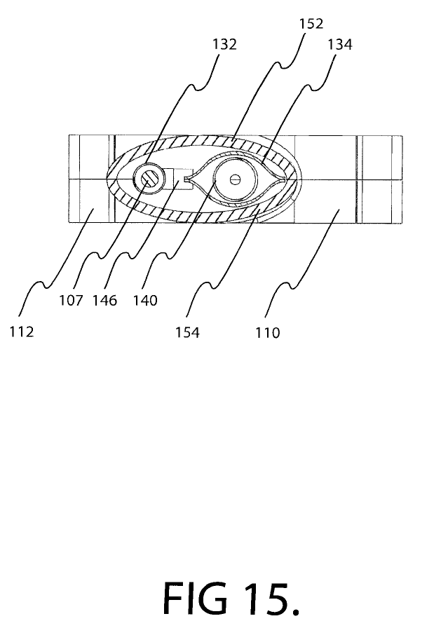

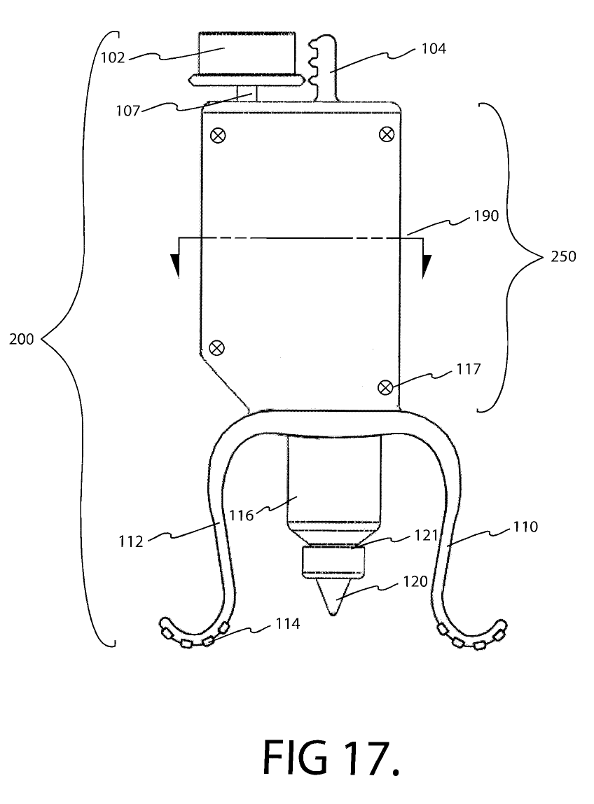

FIG. 15 shows a top section view as defined by section line 190 in FIG. 17, housing halfs 152, 154 can be clearly seen as well as collapsible bladder 134 and pump tube 140. Push foot 146 can be seen as well as piston 107.

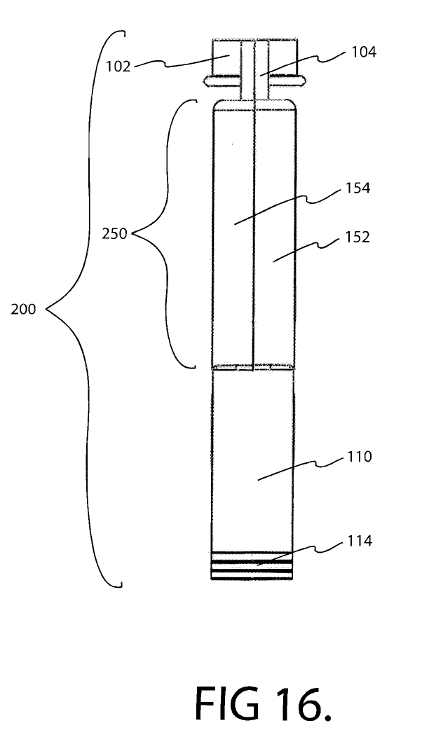

FIG. 16 is a side view of the alternate view 200 of the first embodiment of the invention. Housing halves 152, 154 can be clearly seen as well as indicator post 104 and push button 102. Resilient legs 110 are similar to the legs in all other embodiments of the invention as well as traction strips 114.

FIG. 17 is a front view of the alternate version 200 of the first embodiment of the invention. Screws 117 hold housing halves together. Threaded cap 120 can be clearly seen below exit cylinder 116.

FIG. 18 shows a top view of the alternate version 200 of the first embodiment of the invention. Push button 102 can be clearly seen as well as indicator post 104.

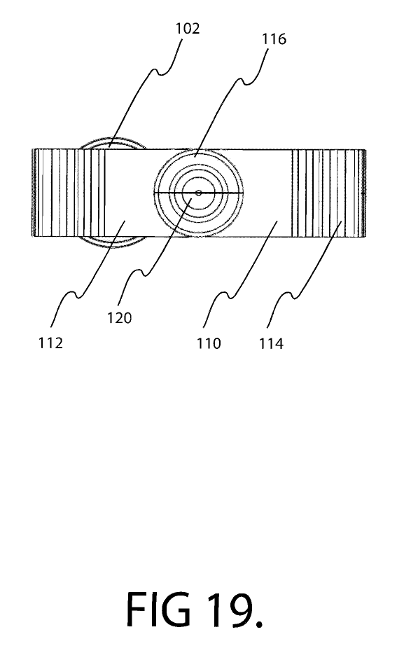

FIG. 19 is a bottom view of the alternate version 200 of the first embodiment of the invention. Traction strips 114 are similar to all other embodiments of the invention.

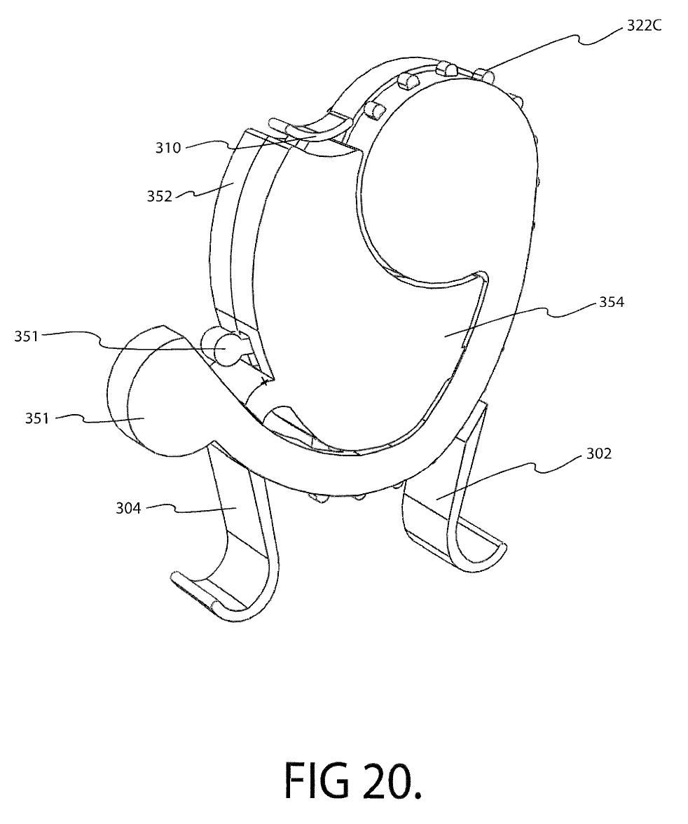

FIG. 20 shows a second embodiment 300 of the precision eyedropper of the present invention. In this version, a cartridge 380 is removable and replaceable from housing 352, 354. Resilient legs 302, 304 work in a similar way as other versions described above and below. To operate, the user presses on push button 310 to expel a precise amount of sterile eye drop solution. The cartridge 380 contains a plurality of discrete solution holding chambers or ampoules 322 as will be described in detail below. By pressing on lever 351, the user advances the next cartridge to the dispensing position and rolls the previously used cartridge into storage area 351.

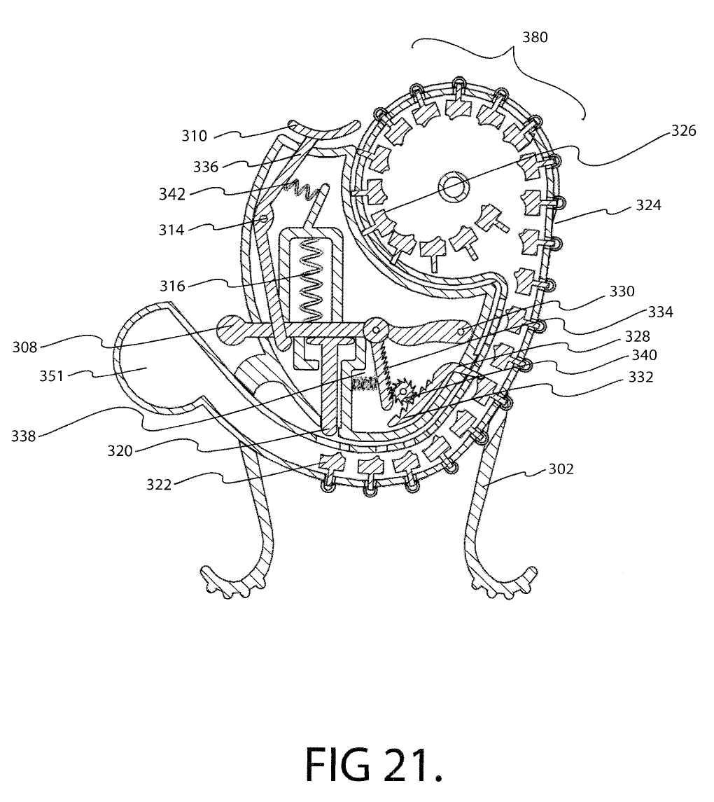

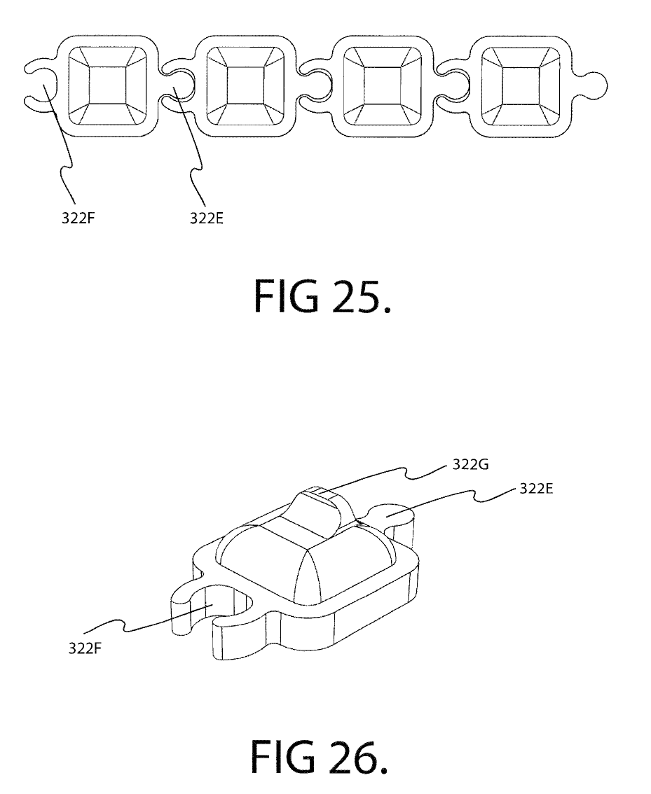

FIG. 21 shows a front section view of the second embodiment of the invention 300. Ampoules 322 are wound up in a snail like pattern. They are each removably attached to each other as shown in FIG. 25. The ampoules 322 are advanced one at a time until they land under the piston member 338. The advancing mechanism works as follows. When the user lifts up on pinned 330 ever end 308, ratchet arm 334 causes pinion gear 340 to rotate which in turn causes second ratchet arm 332 to be pulled down thereby forcing the curved tip of the second ratchet arm 332 to pull an ampoule 322 down towards the push piston 320. When the lever arm 308 is fully lifted spring 342 biased activation arm 336 becomes locked in the up position when the bottom tip of the arm engages the lever 308 and holds it, and the piston 320, in the ready position. When the user presses on the release push button 310, the pinned lever arm 308 is released and forced downward by spring 316, and forcing piston 320 down onto ampoule 322 and squashing it to cause the solution contained within the chamber to be expelled out and into a person's eye. Before an ampoule 320 is used, the user removes cap 322C. Because each ampoule is capped, the solution remains sterile until just before use.

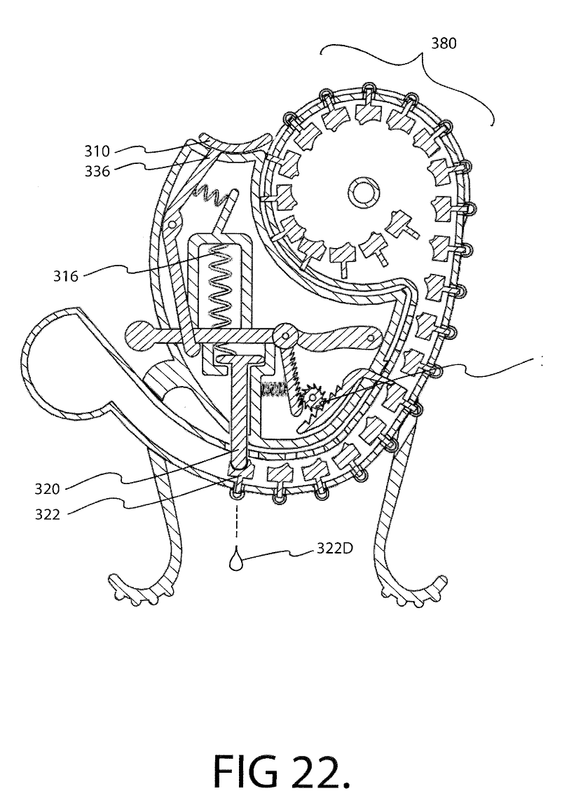

FIG. 22 shows the second embodiment 300 of the present invention in the use mode. Piston 320 has pressed down on ampoule 322 to expel solution drop 322D.

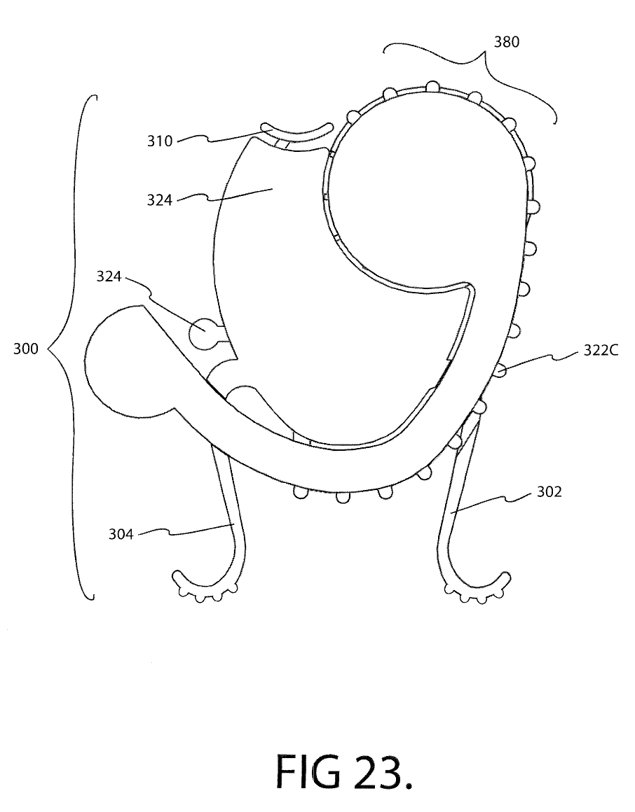

FIG. 23 is a front view of the second embodiment of the invention 300. The caps 322C of each chamber can be seen as they make their way to the point where the piston can compress them.

It is important to note that even though this embodiment shows a plurality of ampoules 322 being stored within a cartridge, an embodiment can also be conceived where only one ampoule 322 at a time is inserted into a delivery device by a user, thereby eliminating the need for a main cartridge and an ampoule advancing mechanism.

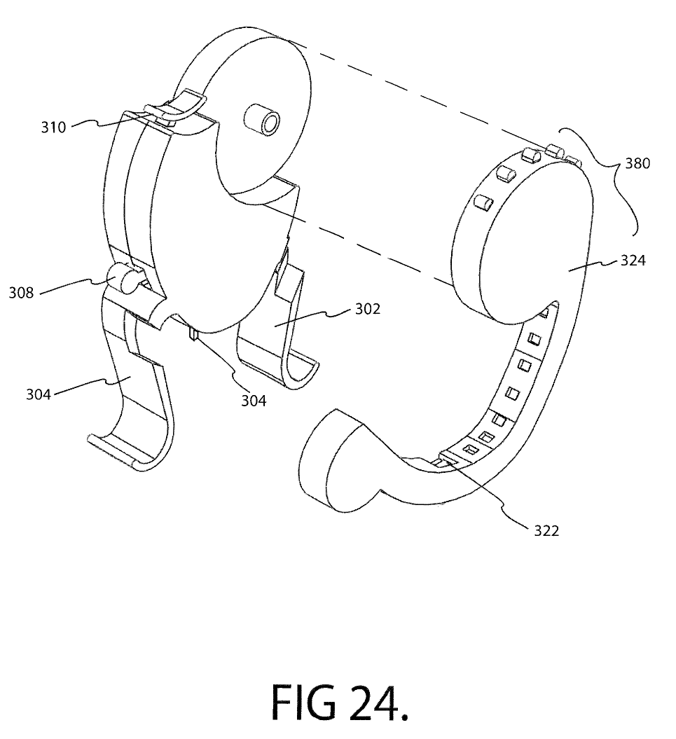

FIG. 24 shows a perspective exploded view of the cartridge version 300. The cartridge 324 can be removed and replaced as necessary.

FIG. 25 shows a group of ampoules 322 that are connected by nodes 322E and C shaped node retainers 322F. Each ampoule 322 includes an upwardly facing tab 322G as shown in FIG. 26 which engages with the curved ratchet member as described earlier, to advance the ampoule one position for each lever arm movement.

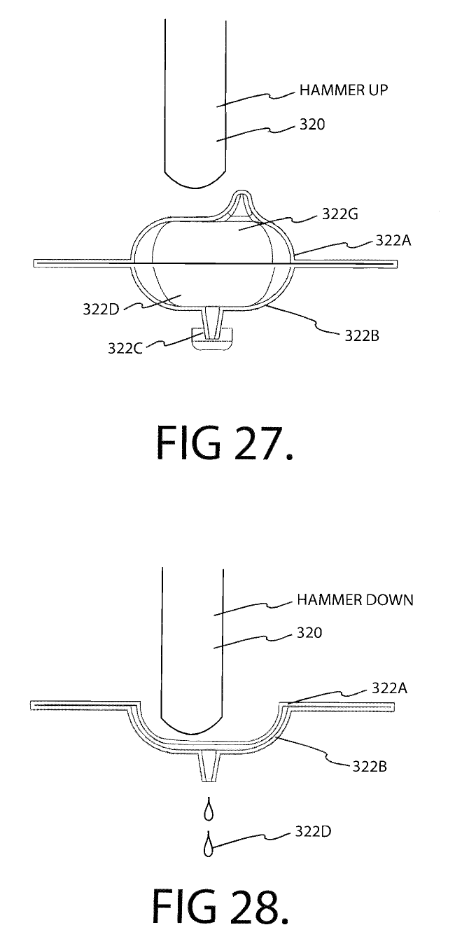

FIG. 27 shows a side view of an ampoule formed of a top half 322A and a bottom half 322B which is filled with solution. FIG. 28 shows the same ampoule after the piston tip 329 has squashed the ampoule half 322A forcing the solution 322D out of exit aperture 322F.

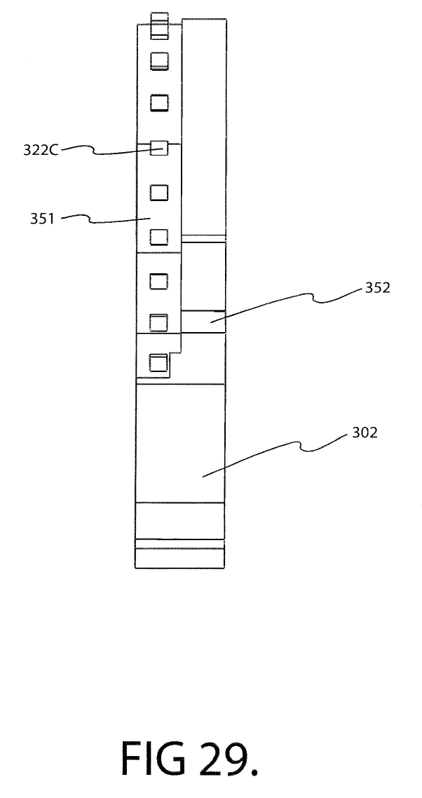

FIG. 29 shows a side view of the second embodiment 300 of the invention.

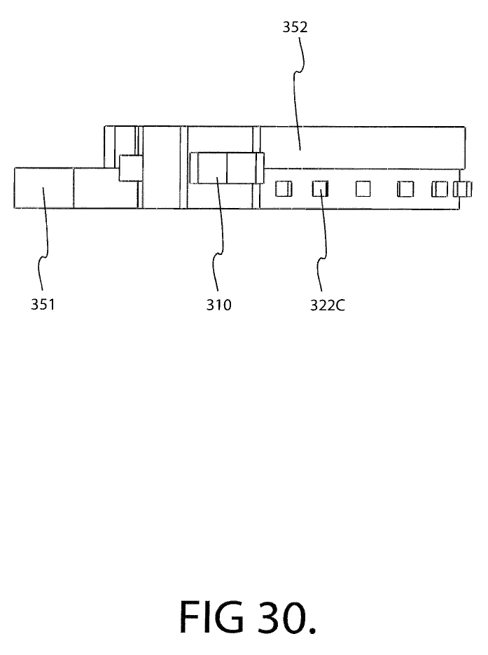

FIG. 30 shows a top view of the second embodiment 300 of the invention

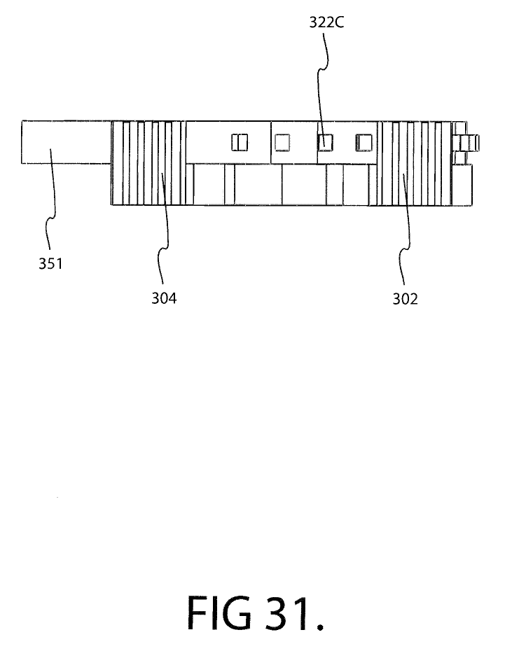

FIG. 31 is a bottom view of the second embodiment 300 of the invention.

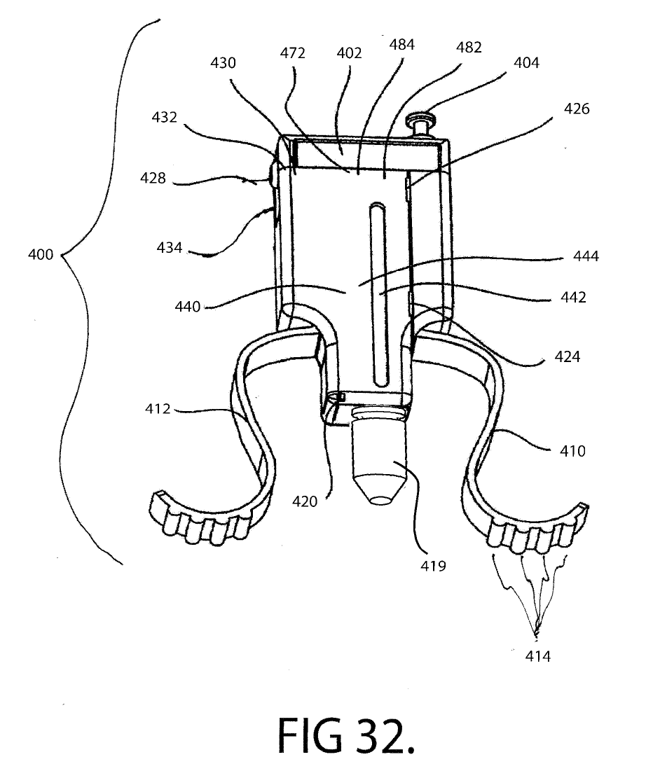

FIG. 32 is a perspective view of a third embodiment of the invention 400. Front door 440 includes an elongated aperture 442 that allows the user to see the amount of solution 450 left in the cartridge 442 that resides inside the housing. Downwardly facing legs 410, 412 and friction strips 414 operate in a similar fashion to the previous eyedropper embodiments described above. The release of drops by pressing push button 404 will be described in detail below. Tip cover 419 protects the tip from damage and dirt during storage.

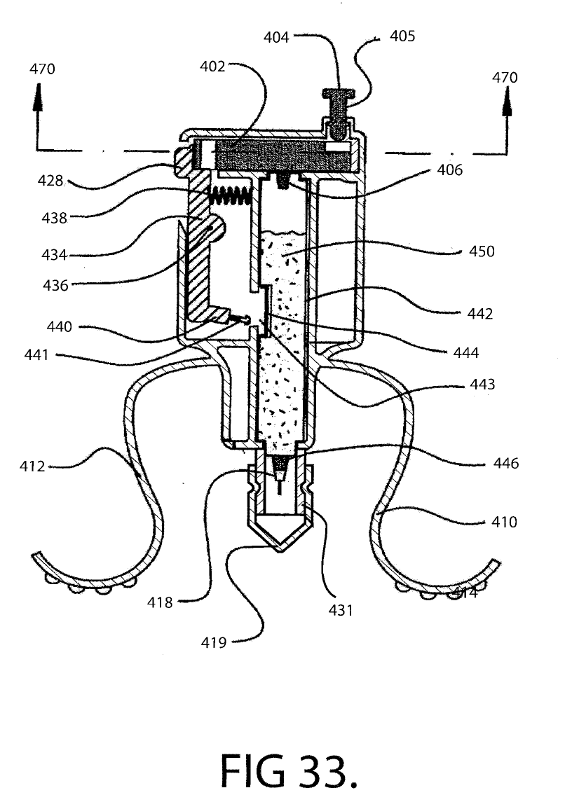

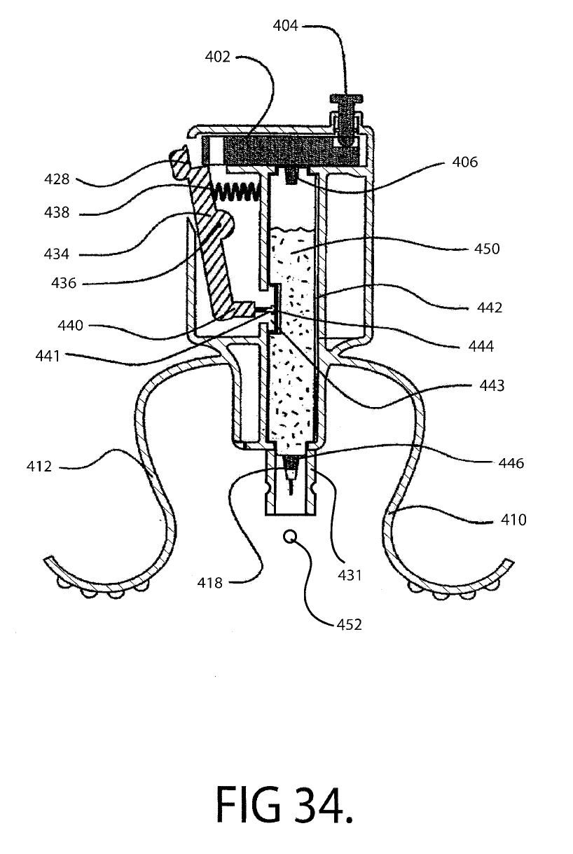

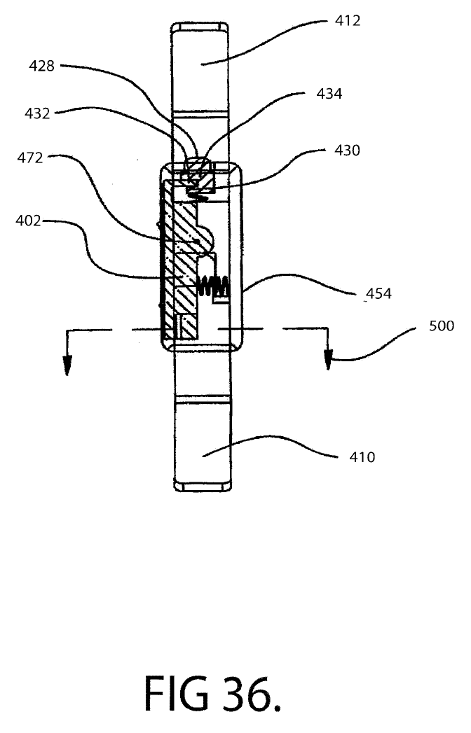

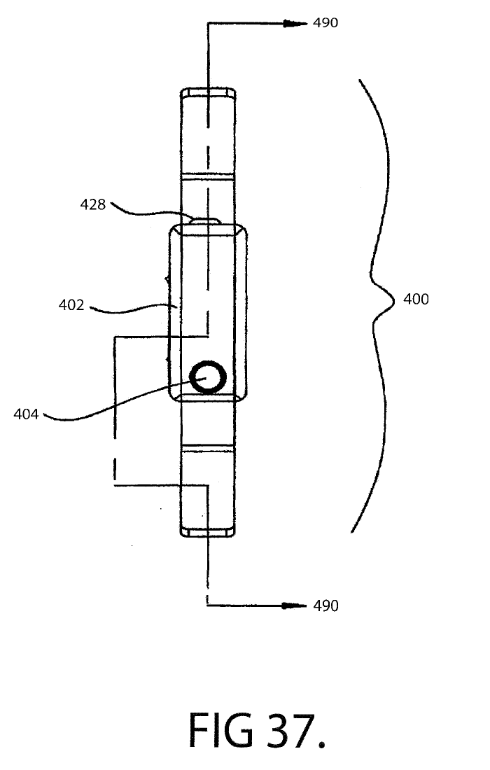

FIG. 33 is a front section view of the third embodiment 400 of the invention as defined by section line 490 in FIG. 37. Cartridge 442 can be seen in section view. The cartridge 442 is elongate and in the preferred embodiment cylindrical. The cartridge 442 is capped on top by top cover 406 that includes an air intake check valve. Cartridge 442 terminates at its bottom in tubular exit aperture 418. A removable cone shaped cap 419 protects aperture 418 and lower cylindrical portion 431 from dirt during non-use. An outwardly directed check valve 446 is mounted just before exit aperture 18. A resilient membrane 444 is fixed over an aperture 443 located in the side wall of cartridge 442. L shaped arm 434 pivots about post 436 and is spring biased 438 to push the bottom portion 440 of L shaped arm 434 into the resilient membrane 444. The thread tip portion 441 of bottom portion 40 is screw threaded into bottom portion 440 thereby allowing the user to adjust the distance between the tip 441 and the membrane 444. As the liquid 450 inside the cartridge 442 decreases in volume, the amount of drops dispensed per use out of aperture 418 can decrease because the remaining air inside cartridge 442 is more compressible than the liquid 450. Therefore the user can adjust the tip 441 by turning it counter clockwise to shorten the distance between the tip 441 and the membrane 444 causing the tip 441 to impinge more deeply into membrane 444 and forcing more drops to dispense per usage. The L shaped arm 434 is held in its potential position by a hook 430, shown in FIG. 36 that engages a mating hook 432, also shown in FIG. 36, on the end of L shaped arm 434. When the user pushes button portion 404, it releases the L shaped arm 434 causing tip 441 to push into resilient membrane 444 as shown in FIG. 33. The resulting deformation of membrane 444 causes air pressure to bear down on solution 450 causing a precise amount of solution to be expelled 452 from aperture 418. Aperture 418 is a narrow diameter such as twenty-six gauge tubing, so that after drop 452 is expelled as shown in FIG. 34, no remaining liquid is left on the tip of aperture 418. Lower cylindrical portion 431 surrounds tip 418 so that the user can not have direct access to it thereby preventing accidental pricking of the finger of the user. When a user pushes on button 428 the membrane 444 returns to its original shape as shown in FIG. 33. This return to flat shape causes a small vacuum to occur in the air portion above liquid 450. This vacuum causes additional air to be drawn into check valve 406 thereby re-pressurizing the air portion above liquid 450.

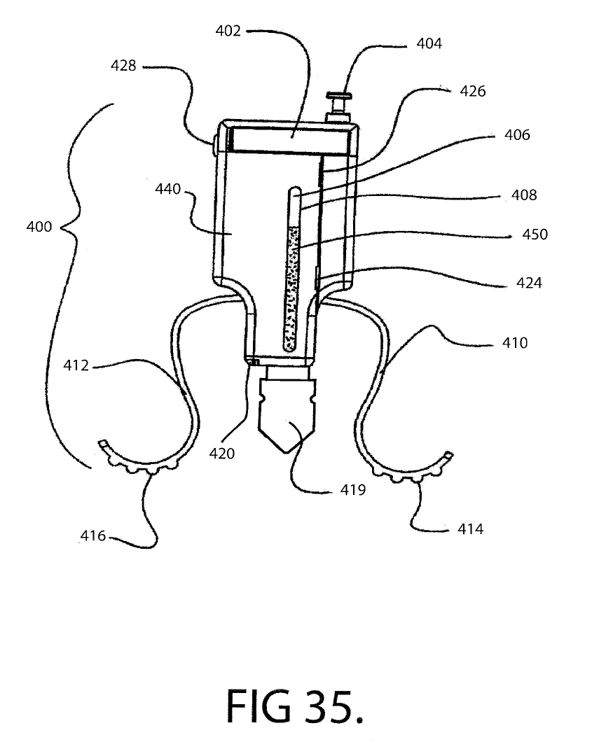

FIG. 35 shows a front view of the third embodiment of the invention 400. Hinge 424, 442 allows front door 440 to swing open to replace cartridge 442. Latch member 420 holds the door shut after the cartridge 442 has been installed.

FIG. 36 shows a top section view as defined by section line 470 in FIG. 33. This view clearly shows spring biased activation arm 402. By pushing down on button 404, arm 402 pivots about shaft 472 causing tab 432 to release tab 430 on L shaped arm 434. The angled termination point of tab 430 allows the L shaped arm 434 to be pushed back to its resting place when button 428 is pushed.

FIG. 37 is a top view of the third embodiment 400 of the invention. Push button 404 can be clearly seen. Section view 490 helps further describe the invention as shown in the section view drawing shown in FIG. 33.

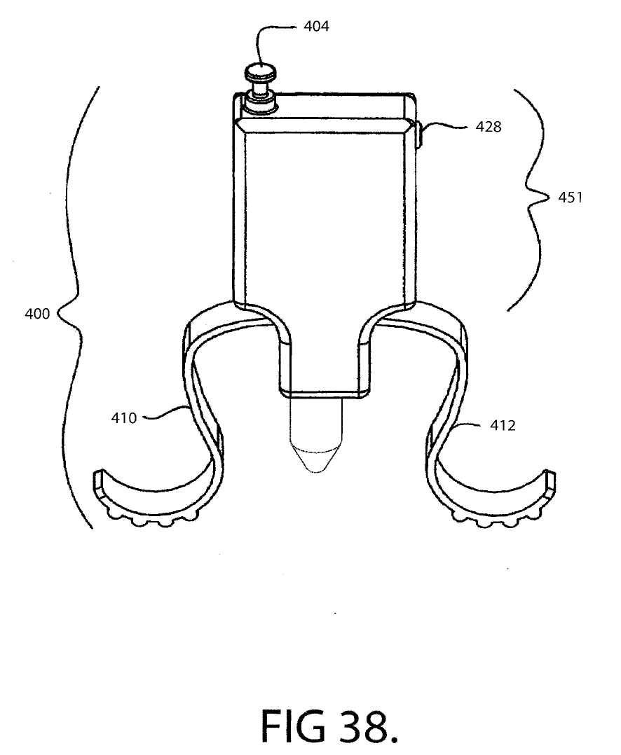

FIG. 38 is a front perspective view of the third embodiment 400 of the invention.

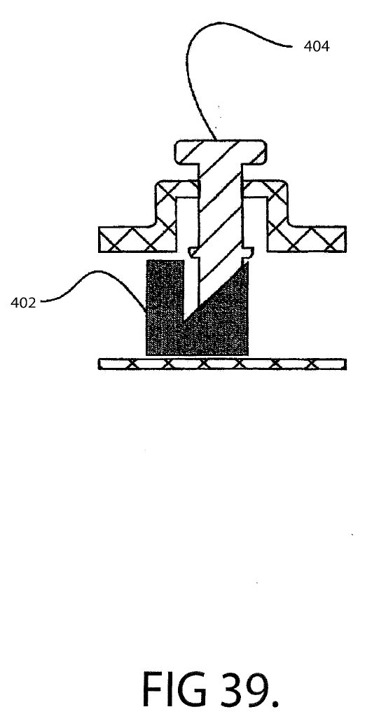

FIG. 39 shows a partial section view of the top push button 404 as defined by section line 500 shown in FIG. 36, and its relationship with activation arm 402. In the armed position, push button 404 and its attached post 405 slidably engage activation arm 402 at the top edge of a V shape 407 in activation arm 402. When the user pushes down with one finger onto push button 404, the arm 402 is caused to move to the right. This movement releases the e L shaped arm 434 causing a drop of solution to exit from the discharge orifice 418.

It should also be noted that another version of the present invention can be modified to act as a sterile nose drop applicator. In this version, the flexible downwardly facing legs shown in the above designs would be eliminated. In the nose drop version, a piston would push down on an ampoule similar to that shown in FIGS. 27 and 28 thereby dispensing a precise amount of nose drop solution into the user's nostril.

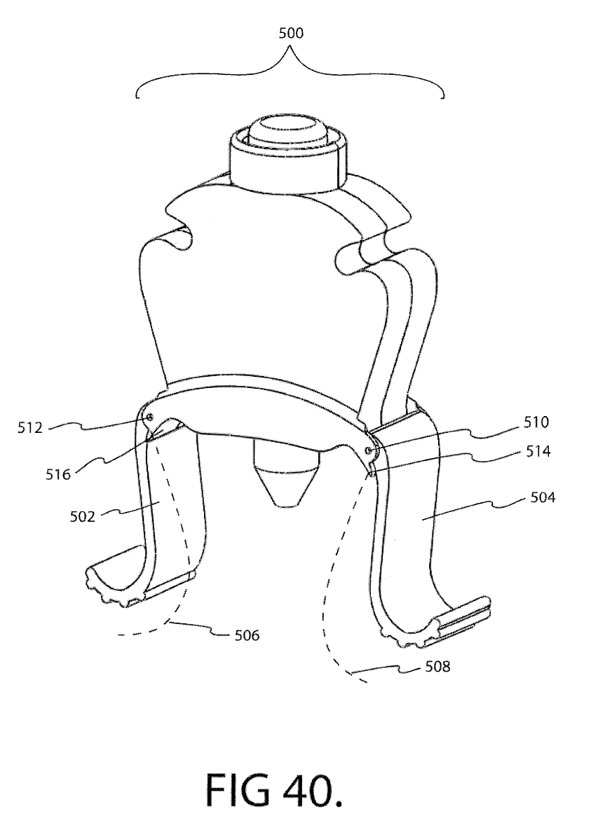

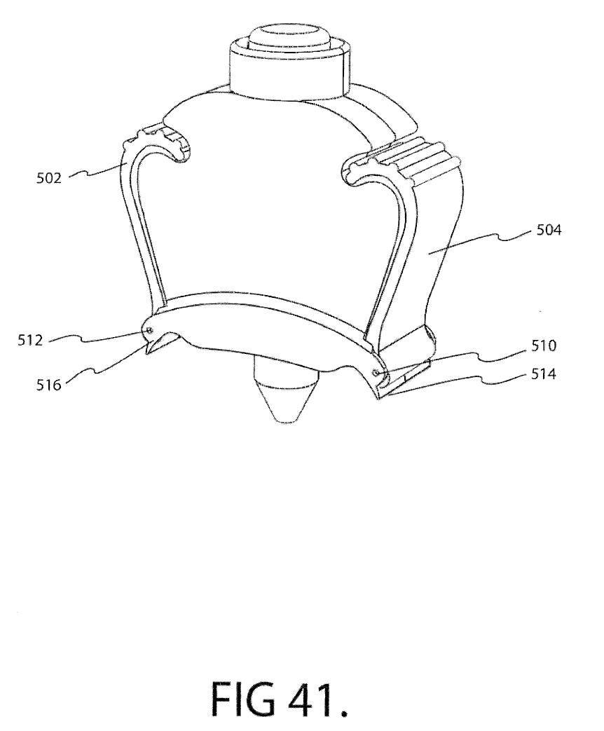

FIG. 40 shows another embodiment of the present invention 500 whose legs 502, 504 are hinged by hinge pins 510, 512 so that they can be folded up one hundred and eighty degrees as shown in FIG. 41. The folded up position allows the device 500 to be stored or packaged in a smaller space thereby saving in shipping and storage costs. Leg stops 514, 516 support the upper portion of the legs 502, 504 so that when the legs 502, 504 are squeezed inward as shown by dotted lines 506, 508 the resilient nature of the plastic legs allows them to move toward each other in a spring biased fashion as they resist the leg stops 514, 516.

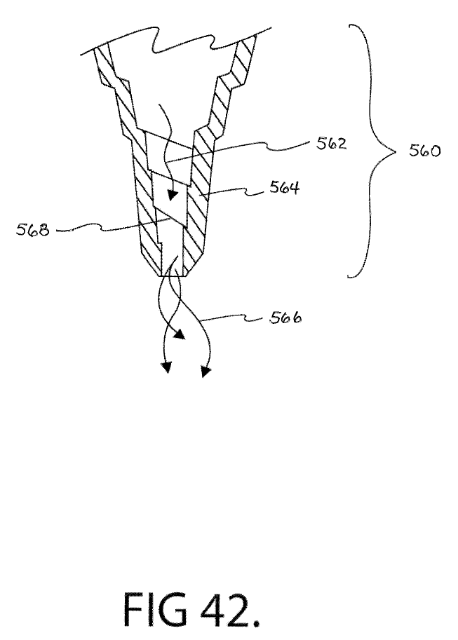

FIG. 42 shows a partial section view of a solution dispenser tip 560 that can be used as a tip on any of the above described eye drop dispensing devices. The tip end 564 includes an internal wall architecture that includes a spiral pattern 568 that causes the outgoing eye drop solution to swirl as indicated by direction line 562. The spiraling effect produces a spray pattern when the solution exits the tip as indicated by direction lines 566. Other standard spray aperture designs can also be used to produce a spray pattern. A spray effect may be superior to a propelled droplet configuration because a spray hits the lower sclera area of the conjunctiva coating of the eye with considerably less force thereby reducing the chance of damage to the conjunctiva coating.

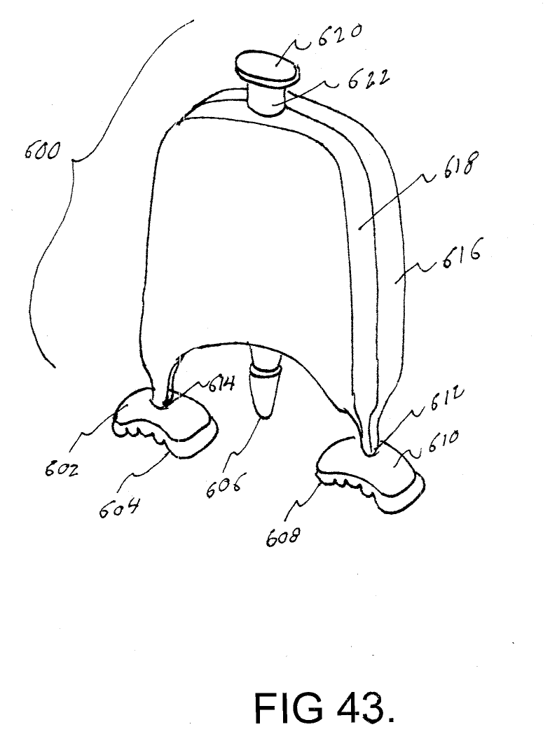

Referring now to FIG. 43 we see a perspective view of the fourth preferred embodiment of the eye dropper invention 600. This embodiment does not employ the same eye spreading mechanism that has been shown in earlier versions such the version seen in FIG. 1 and labeled as 12 and 14. In the version shown in FIG. 1 the user squeezes on legs 10 and 12, then places the bottom portion of the legs on the upper and lower portion of the orbital socket, and then releases the formerly squeezed legs so that they spread, causing the eye lids to be forced open. In the preferred embodiment shown in FIG. 43 the legs have no resilient element built in. Instead, the user uses one hand to hold the device 600 and place one foot portion 602 on the lower orbital socket. Then, with the other hand, the user spreads his or her lid to the wide open position and, while in that position, places the other foot 610 down onto the upper portion of the orbital socket thereby holding the lids in a spread condition. The advantage of this type of operation is that there is no danger of the user forcing his or her eyelids to open wider than is comfortable because the user is in full control of the amount of opening he or she desires. The bottoms of the feet 604, 608 are over molded with a soft rubber like plastic for maximum comfort for the user. The feet are capable of pivoting slightly because of the slim, flexible junction of the feet 602, 610 to the leg at area 614 and 612. The feet are shaped in a concave manner to help them conform to the upper and lower orbital socket bone structure. An alternate embodiment of the present design 600 can include the concept of foldable legs as has been described in an earlier embodiment 500 shown in FIG. 38.

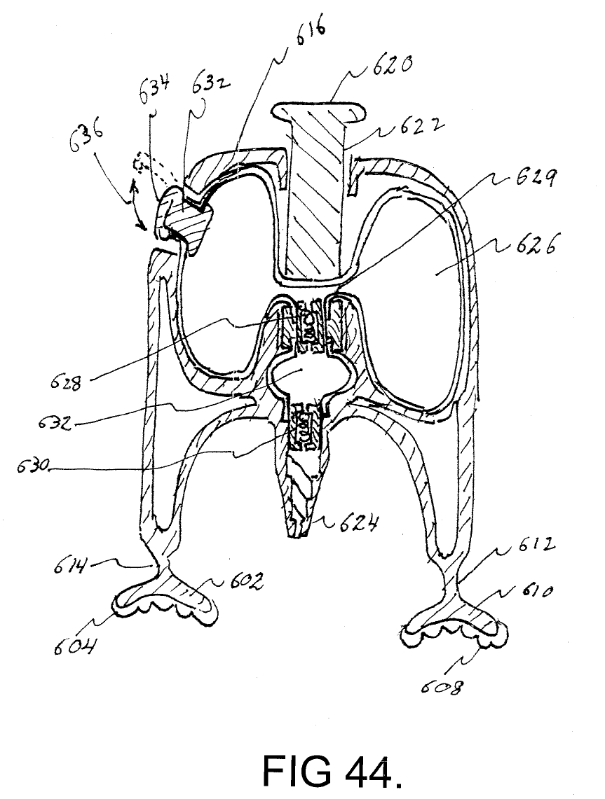

A hollow housing is comprised of a front half 618 and a rear half 616 encloses a bladder and dispensing mechanism as will be discussed below. The user removes dispensing tip cover 606 before use and replaces it after each use. After the device 600 is in place over the user's eye, the user presses down on the actuator button 620 causing shaft 632 to lower and force a precise amount of liquid out of the dispensing tip as will be shown in the following FIG. 44.

FIG. 44 shows a front section view of the fourth preferred embodiment of the invention 600 showing the inside of hollow housing halves 616, 618. A collapsible bladder 626 can retain eye drop solution. The bladder 626 is doughnut shaped allowing the centrally disposed plunger shaft 622 to extend through the central portion of the bladder and down to the one way valve assembly 628. A tubular portion 629 connects the main bladder 626 with the first one way valve 628.

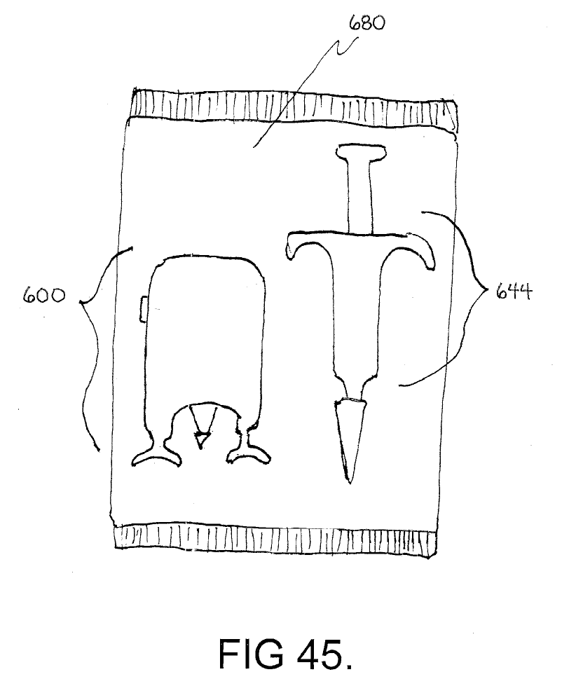

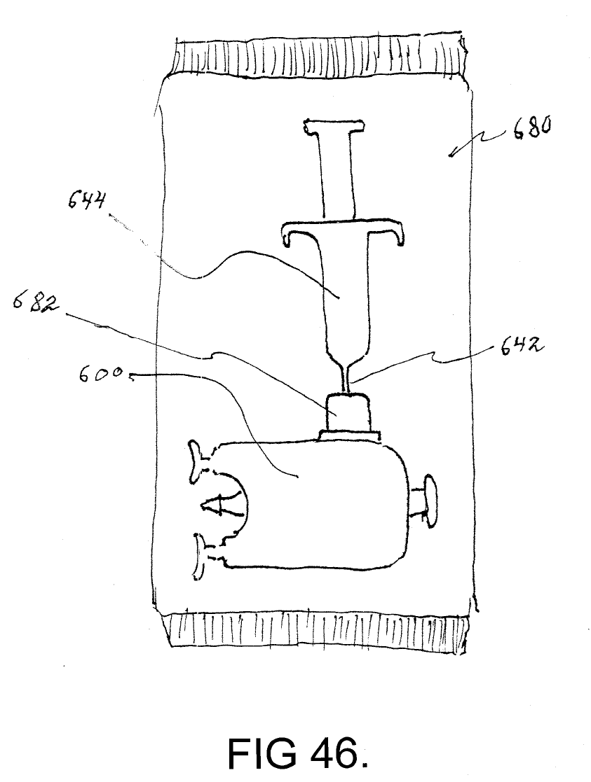

Eye drop solution is injected into the bladder through rubber stopper 632. The user lifts on flap 634 as shown by direction line 636. The user then removes a syringe 644 from a sealed plastic bag 646 as shown in FIG. 45. The user removes protective tip 648 and inserts the tip through the rubber stopper 632 as shown in FIG. 46. The tip 638 is closed off to prevent loose portions of rubber of stopper 632 from entering the tip and contaminating the solution. The eye drop solution is injected through side aperture 640 into the bladder area 626. Then the syringe tip 536 is removed from the rubber stopper 632 causing the aperture formed in the stopper 632 to seal shut. This method of filling ensures that the eye drop solution remains in a sterile state until dispensed by the user during an eye drop application event. To further insure a sterile state, a sleeve, not shown, can surround the tip of the syringe needle and can be slid back in the process of inserting the needle tip 638 into the rubber stopper 632. In this way, there is no chance for the needle or solution to be contaminated and become non-sterile.

Referring back to FIG. 44, when the user presses down on push button 620, the attached shaft 622 it causes eye drop solution to be drawn from the main bladder 626 through a first one way valve 628 and into single portion bladder 632. Thereafter, each time the push button 620 is pressed a single portion of solution is forced into the single portion bladder and out through a second one way valve 630 and finally out the exit aperture 624 and into the user's eye. Because the main bladder 626 is constructed of very flexible material, such as thin latex such as found in condoms, the main bladder 626 contracts, thereby not allowing any air to enter the bladder. Exit aperture 624 includes an internal spiral construction similar to the construction described in FIG. 42, to cause the eye drop solution to be expelled as a spray. The embodiment shown in FIGS. 43 and 44 is preferred because of its simple construction and its sterile application.

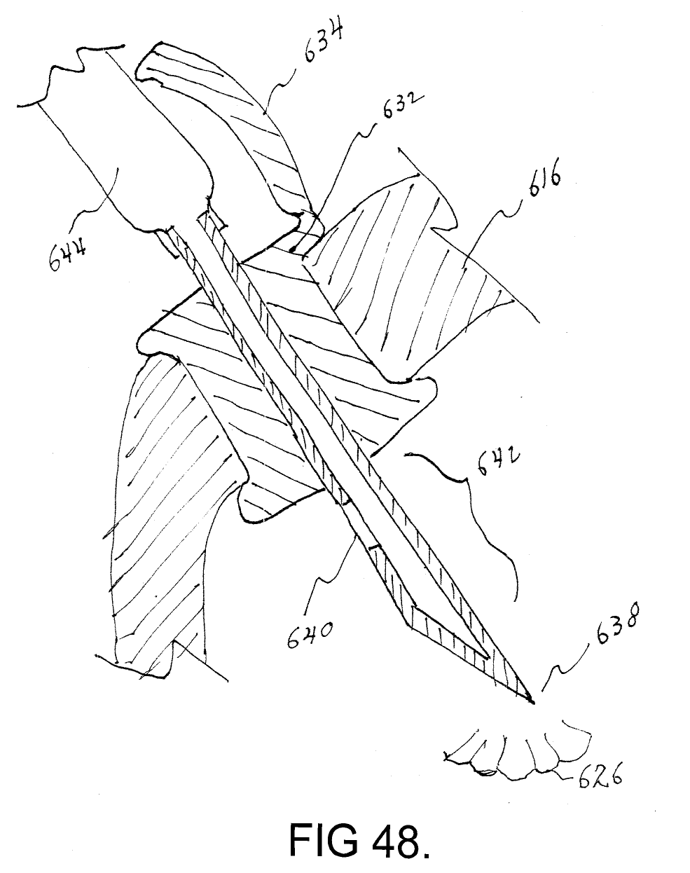

To further promote a sterile environment, FIG. 45 shows the embodiment 600 packaged in an air tight bag 680 along with a syringe 644 filled with eye drop solution. The user opens the bag, and then injects the eye drop solution into the device 600 as shown FIG. 48 in a partial perspective view. This view shows the needle 642 is closed at the end 638 and has a side aperture 640. The user pushes the needle 642 into and through rubber stopper 632 and then injects the eye drop solution from barrel 644 and into bladder 626. In this way, there is no chance of coring or crumbling during the needle insertion process thereby eliminating the possibility of rubber debris entering bladder 626.

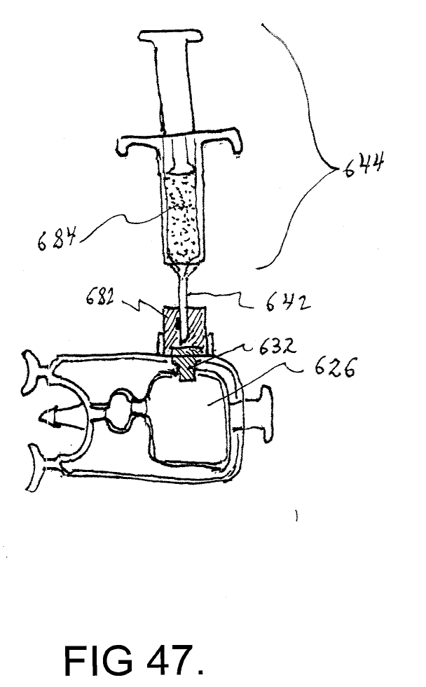

FIGS. 46 and 47 show another packaging design to further insure a sterile embodiment of the device 600 and the syringe 644. The device 600 and syringe 644 are packaged in an air tight bag 680. In this embodiment the needle 642 of the syringe is inserted into a resilient member 682. After the user opens the bag 680, he or she pushes the needle 642 of the syringe through the rest of resilient member 682 and into and through rubber stopper 632 and then injects the solution 684 into the bladder 626 as described in FIG. 48. The resilient member 682 and rubber stopper 632 are made of high grade; forty shore silicone rubber which is FDA approved and has virtually no chance of coring or crumbling during the injection process.

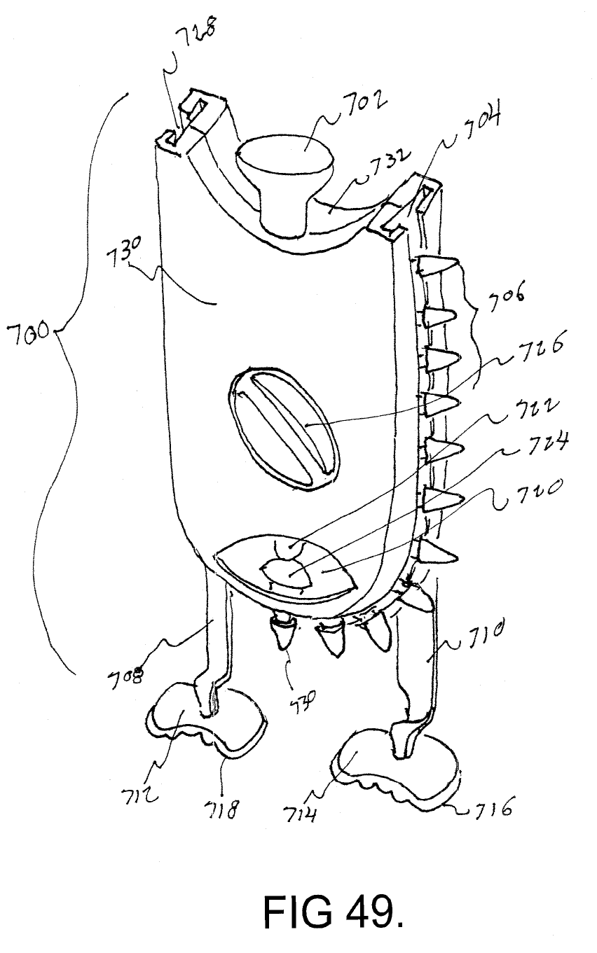

FIG. 49 shows a perspective view of a fifth preferred alternate embodiment 700. This embodiment employs a portion pack strip 706 that is slid into one side of the housing 730, 732 through track 704 so that the portion pack caps 730 extend outward. The lowest cap 730 can be removed by the user just before an eye drop application. The user places the feet 712, 714 of the device 700 onto his or her orbital socket as described in the previous embodiment 600. Thinned leg members 754, 756 allow the feet 712, 714 to flex slightly to accommodate the variety of shapes of orbital sockets. Rubber gripping pads 718, 716 are over molded under feet 712, 714 to help grip the skin and to also provide a soft surface to avoid damage to the delicate skin located just over the orbital bone structure. A transparent window 720 allows the user to see the location of the single portion top 724 in relation to the plunger tip 722. If the alignment is not correct, the user can turn alignment knob 726 to help make minor alignment adjustments.

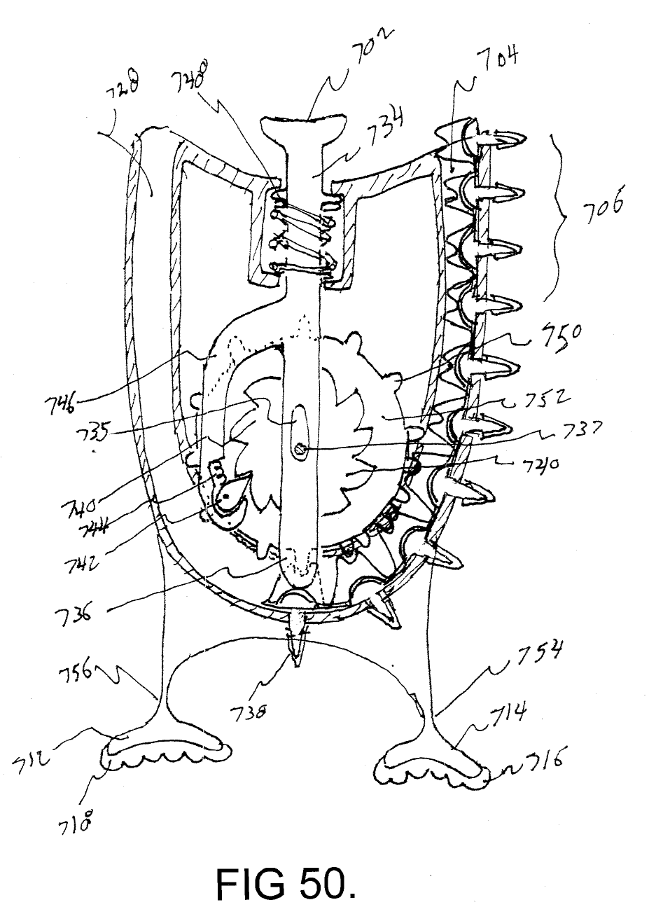

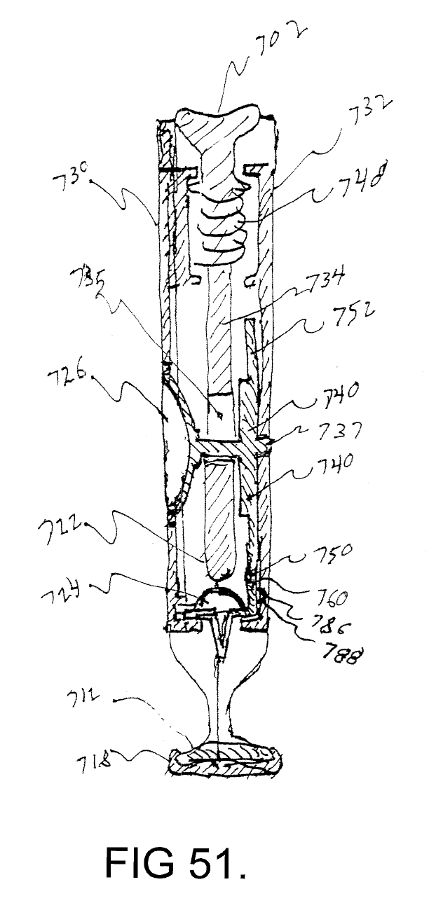

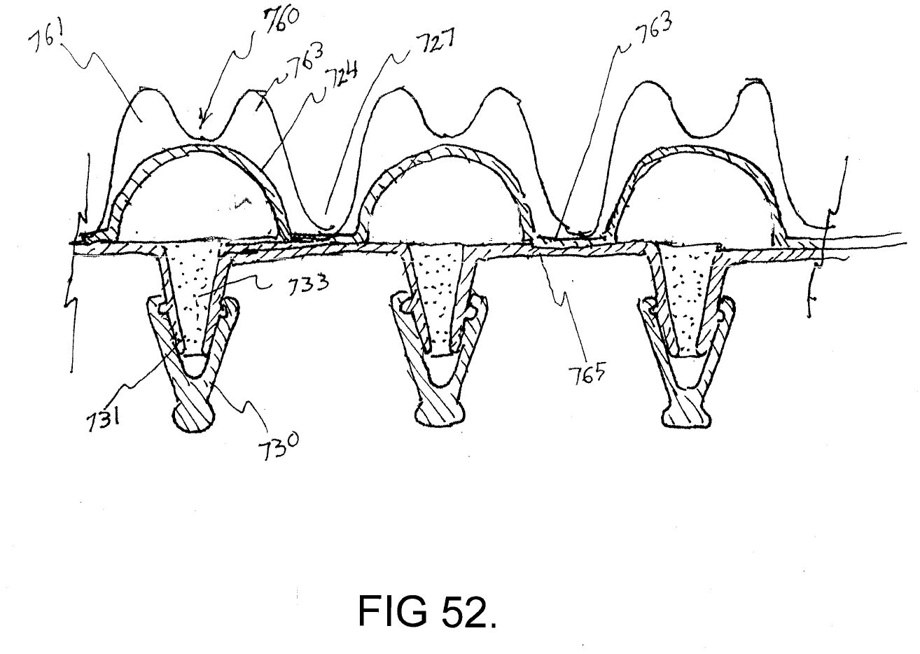

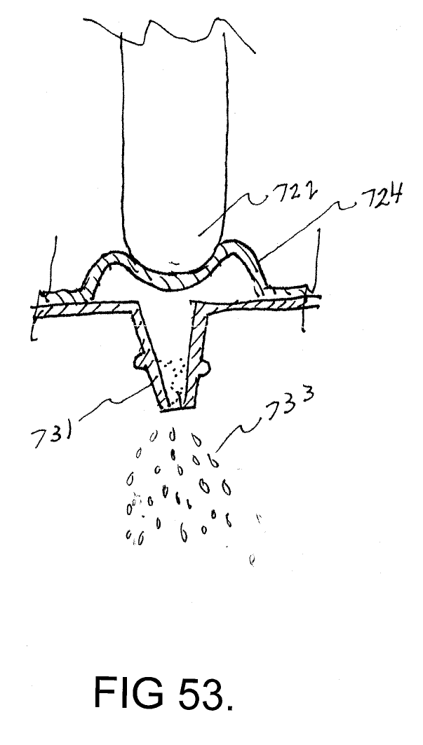

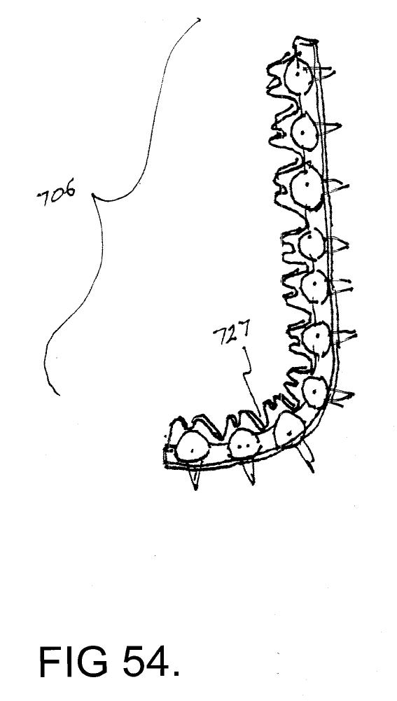

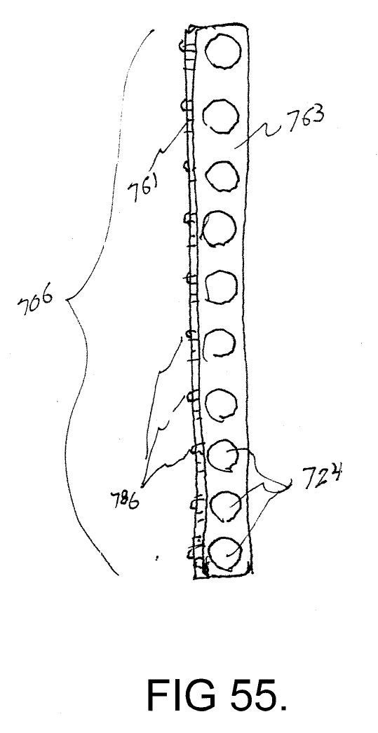

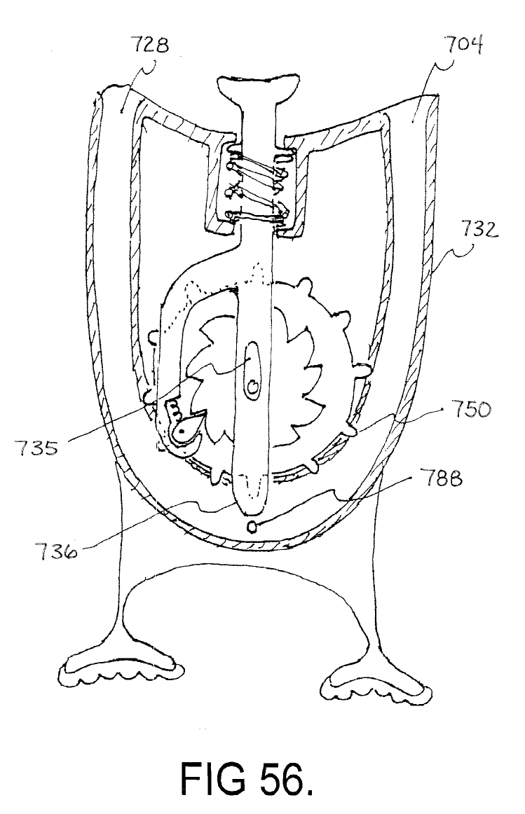

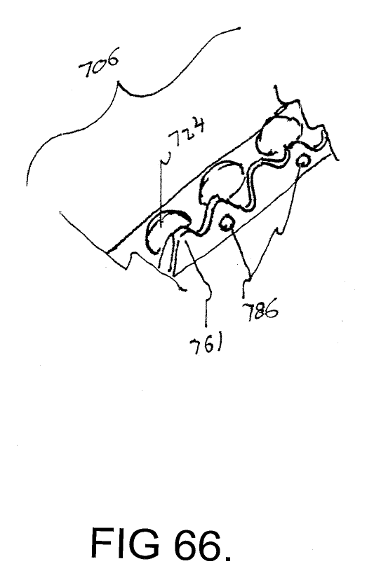

FIG. 50 shows a front section view of the present embodiment 700. This view shows the internal mechanism found inside hollow body halves 730, 732. A strip 706 of portion pack modules is slidably placed inside track 704. A closer look at the construction of the portion packs can be seen in FIGS. 52, 53, 54 and 55. The portion packs are formed by a top strip 763 and a bottom strip 765 as shown in FIG. 53. The top strip 763 has dome shapes 724 formed into it. The bottom strip 765 has cone shapes 731 formed into it. The cone shapes 731 are covered by a removable tip 730. The cone shapes 731 are filled with a single portion of eye drop solution 733. The two strips 763 and 765 are fused together during production. The top strip 763 includes an undulating flange portion that is at right angles to the strip 763. The flange portion includes two raised members 761, 763 and a valley portion 760. In between each flange portion is a deep valley 727 that touches the top surface of top strip 763. On the reverse side of the flange portion, near the peak portion 763 is an outwardly protruding dome shaped bump 786 that can be seen in side section view of FIG. 51 as it engages a mating depression 788 thereby accurately locating the dome shape 724 directly below the plunger tip 722. Dome shaped bump 786 can also be clearly seen in FIG. 55, and FIG. 66. The depression 788 located on the rear wall of the housing half 732 can be clearly seen in FIG. 56. The strips 763, 765 are made of thin resilient plastic such as low density polyethylene. The flexible nature of the strips allows them to flex while traveling through the curved input track 704 and output track 728. The resilient nature of the strips also allows domes 724 to be depressed when plunger tip 736 strikes them as shown in FIG. 54, causing the eye drop solution 731 to be expelled in micro droplets 733 and into the user's eye.

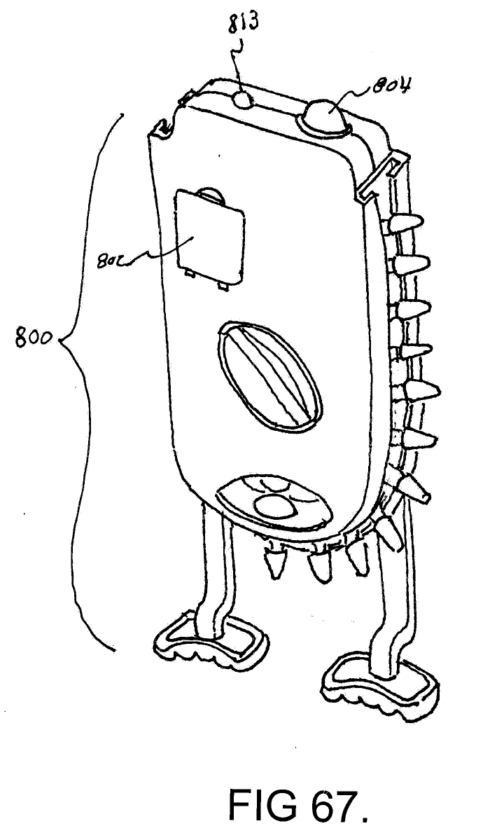

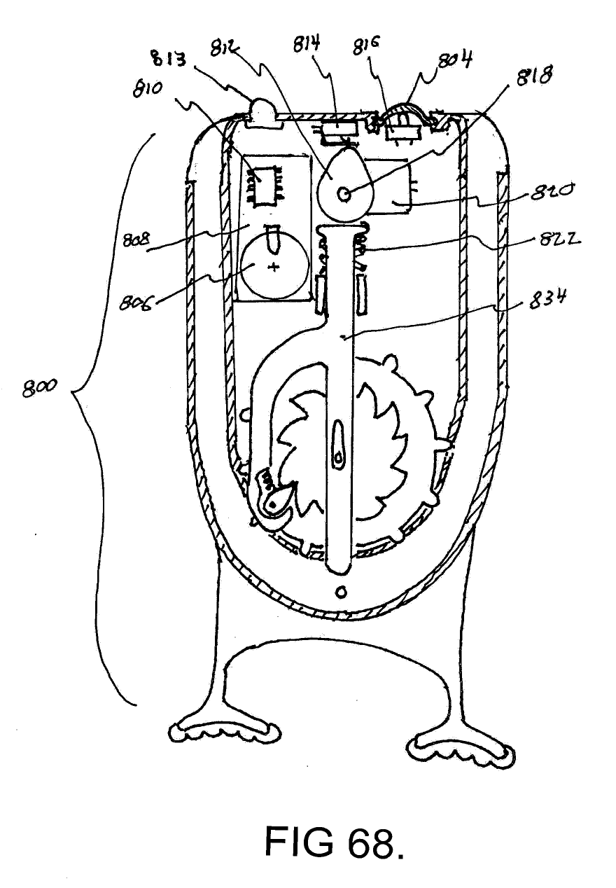

FIG. 67 shows a perspective view of an automated version of the fifth preferred alternate embodiment of the invention 800. This version works in a similar way to the version 700 except that the act of moving the dispensing plunger 834 downward is assisted by an electric motor and gear assembly 820 shown in the front section view of FIG. 68. In this version, the user presses a dispense button 804 causing a momentary electrical switch 816 to be activated. This sends a signal to a microprocessor 810, mounted on PCB 806, which in turn gives instructions to a standard micro motor and gear reduction assembly 820 to activate. A cam 812 is fixedly attached to the output shaft 818 of motor 820. As the cam rotates it pushes down on the upper platform of plunger shaft 834 causing the dome of the portion pack to be depressed as shown in the embodiment described in FIG. 53. Spring 822 causes the plunger shaft 834 to raise as the cam 812 returns to its start position. LED light 813 flashes when a drop is dispensed, giving the user an additional verification that a dispensing action has occurred. When the cam 812 reaches the start position a normally on momentary switch 814 is pushed to the off position by the cam tip thereby turning off motor 820. The device 800 is now ready for the next dispensing event. The battery 806 may be replaced by the user by opening battery door 802.

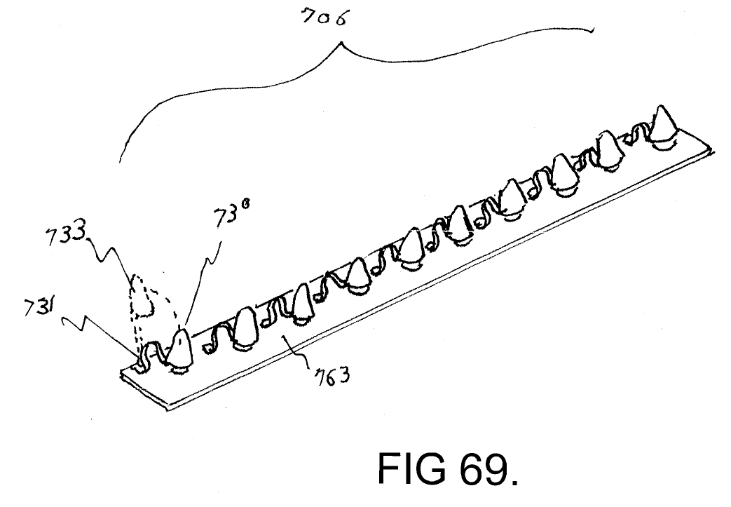

FIG. 69 shows a perspective view of the portion pack strip 706 where the cap portion 730 is retained by a tether 731 so that when the user removes the cap 730 it remains with the strip 763 as shown by dotted line drawing 733.

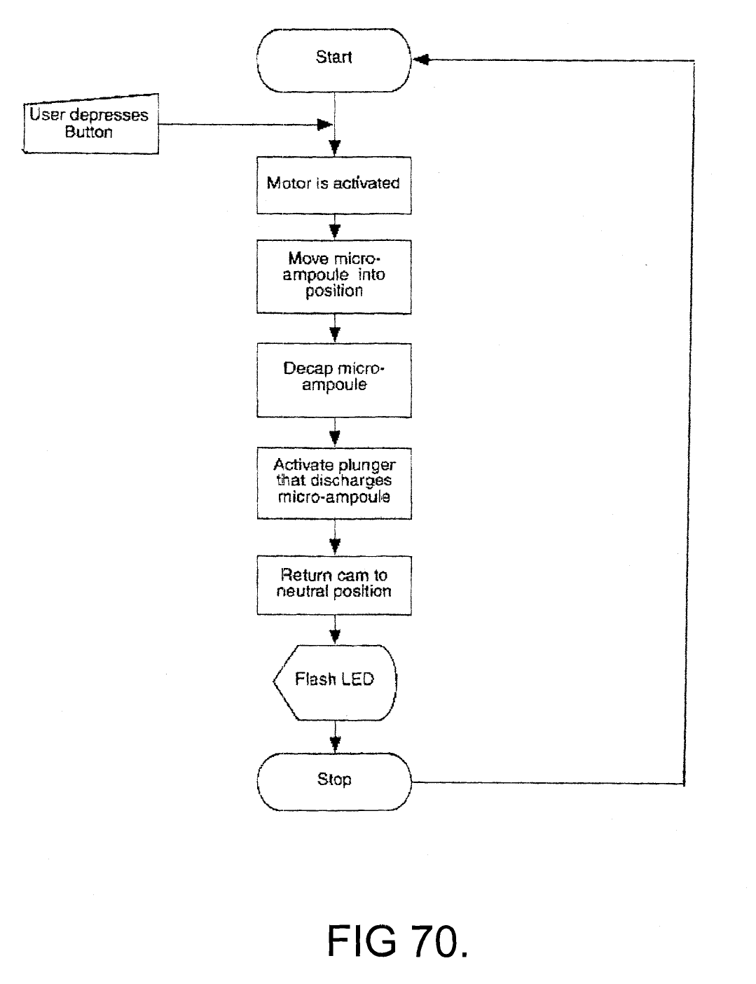

FIG. 70 shows a schematic flow chart of the steps of using the motorized version 800 of the present invention.

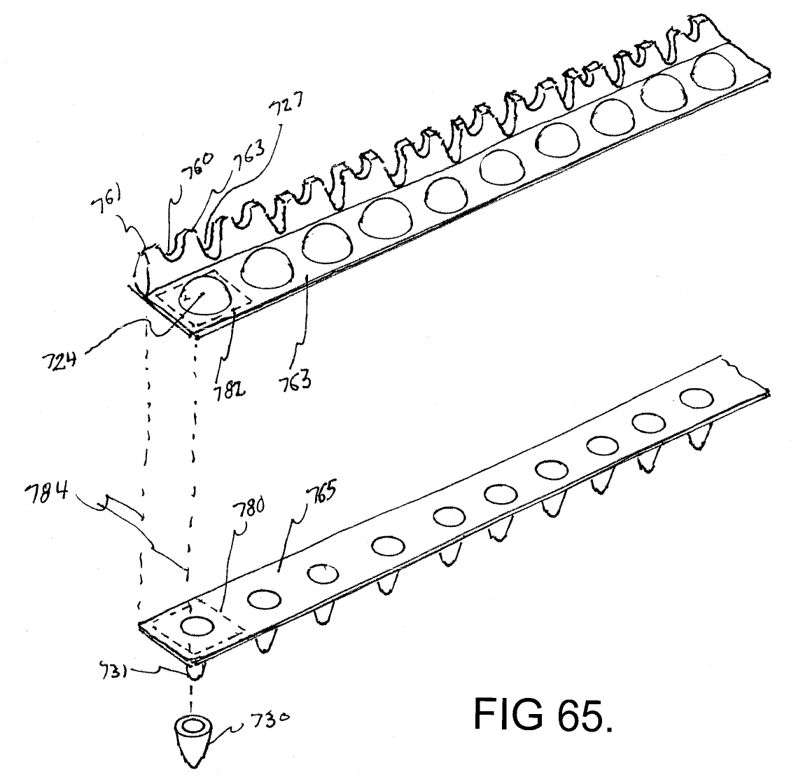

FIG. 65 shows an exploded view of the strip assembly 706. The bottom strip 765 includes evenly spaced tip portions 731. The top strip 763 includes molded in dome portions 724 and undulating flange portion comprised of peak portions 761, 763 and valley portions 760, 727. After the bottom cone shaped tips 731 are filled with solution 733, the two strips 763, 765 are heat welded together at dotted lines 780, 782. This welding process continues in a similar fashion for all the tips 731 and dome shapes 724 of the remaining nine portion pack modules also known as micro-ampoules. Obviously, the entire portion pack strip assembly 706 can be manufactured in varying lengths depending on the number of micro-ampoules intended to be used in each portion pack strip 706.

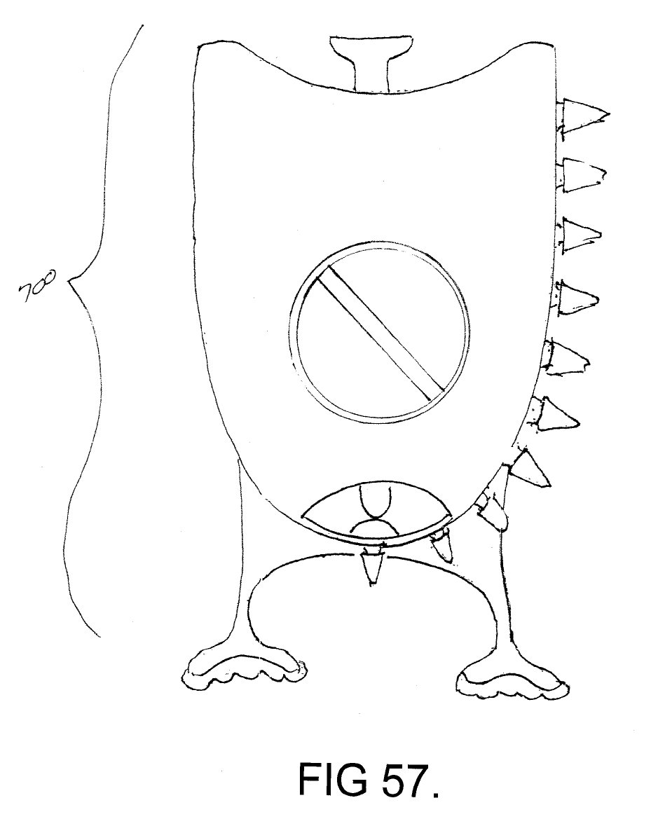

FIG. 54 shows a single strip in a flexed position as it would look when inside the housing 700. FIG. 57 shows a top view of the strip 706 in its straight configuration. Flange portion 761 can be seen as well as circular dome shapes 724.

Referring back to FIG. 50, a portion advancing mechanism causes one portion to be advanced each time the user releases the push button 702 thereby making it ready for the next eye drop application. The advancement mechanism works as follows. During the upward travel of the plunger shaft 734, facilitated by compression spring 748, a pawl 744 pushes up on a tooth of ratchet wheel 740. An attached disk 752 includes a plurality of radially displaced posts 750 that can engage the valley portion 760 of flange 761 thereby driving the entire strip assembly 706 forward one station. Spring 744 causes the pawl 742 to return to its start position. In the above described and illustrated way, the portion pack strip is advanced each time a person uses the device 700. The used strip slides up track 728 and can be pulled out of the top of the track 728 when all the portions have been used up.

FIG. 51 shows a side section view of the present embodiment 700. This view shows that drive disk 752 and ratchet disk 740 and adjustment knob 726 are all fixedly attached to each other. Advancement post 750 can be seen engaging flange valley 760. Plunger shaft 734 includes a slot 735 to allow shaft 737 to pass through.

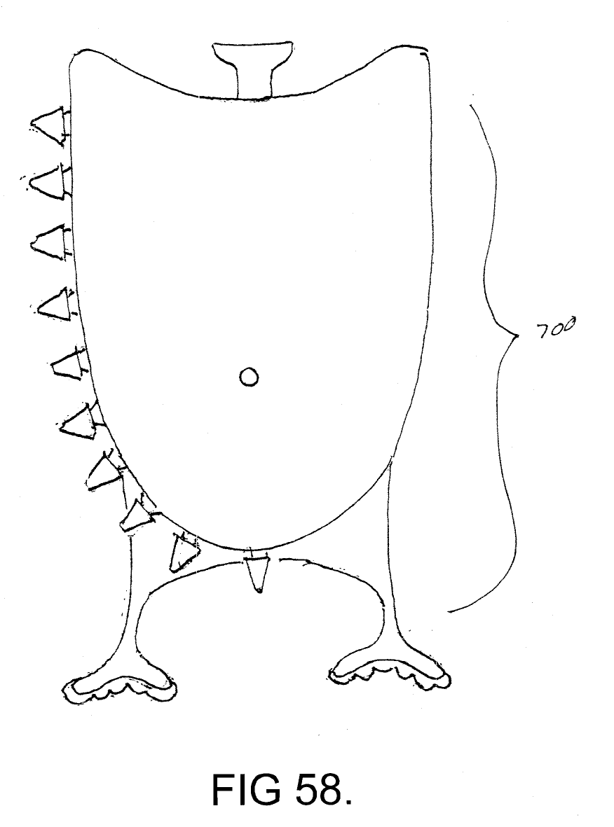

FIG. 56 shows a front section view of the invention 700 with the portion pack strip removed for a clearer view of the advancement mechanism. FIG. 57 is a front view of the present embodiment 700. FIG. 58 is a rear view of the present embodiment 700.

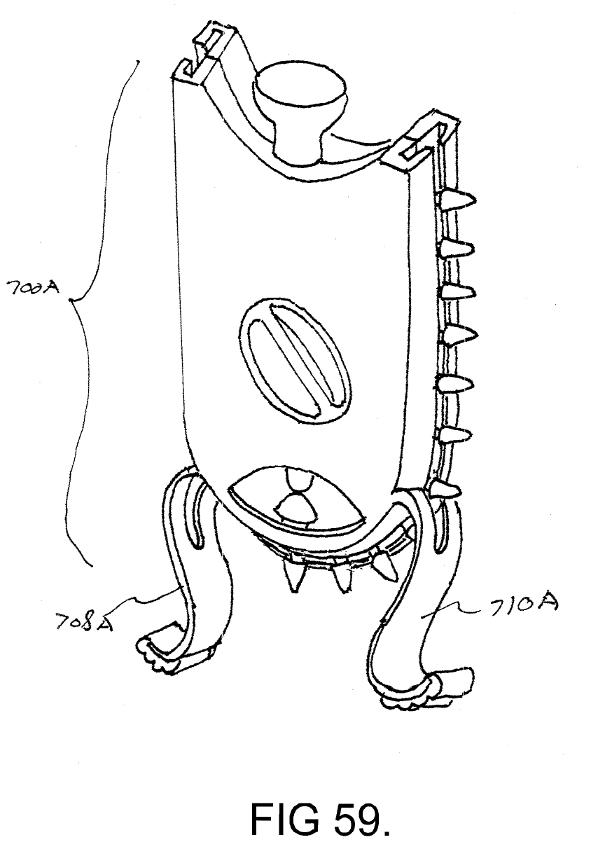

FIG. 59 is a perspective view 700A of the first preferred alternate embodiment of the invention having resilient legs 708A, 710A similar to the ones shown in the first embodiment in FIG. 1.

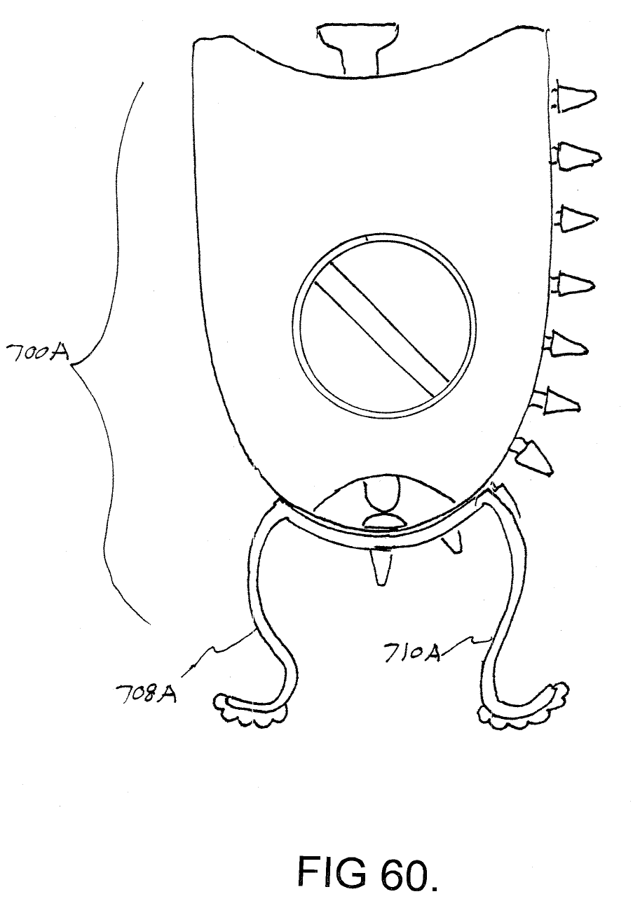

FIG. 60 is a front view of the 700A embodiment.

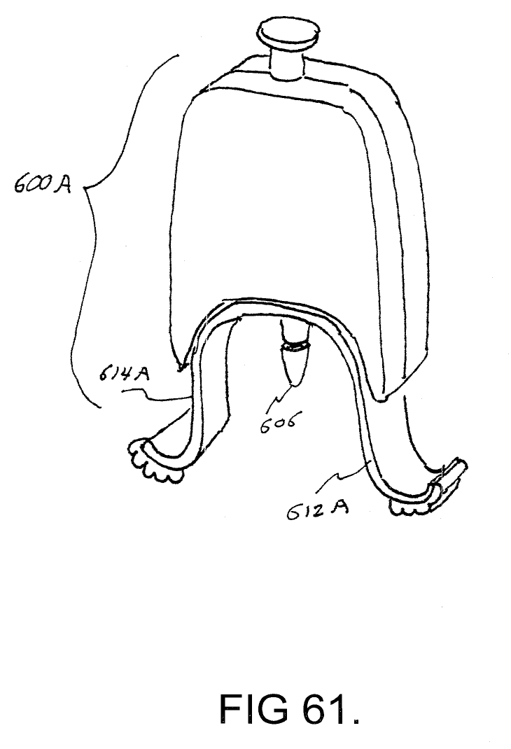

FIG. 61 is a perspective view 600A of a version of the fifth preferred embodiment that has resilient legs 614A, 612A.

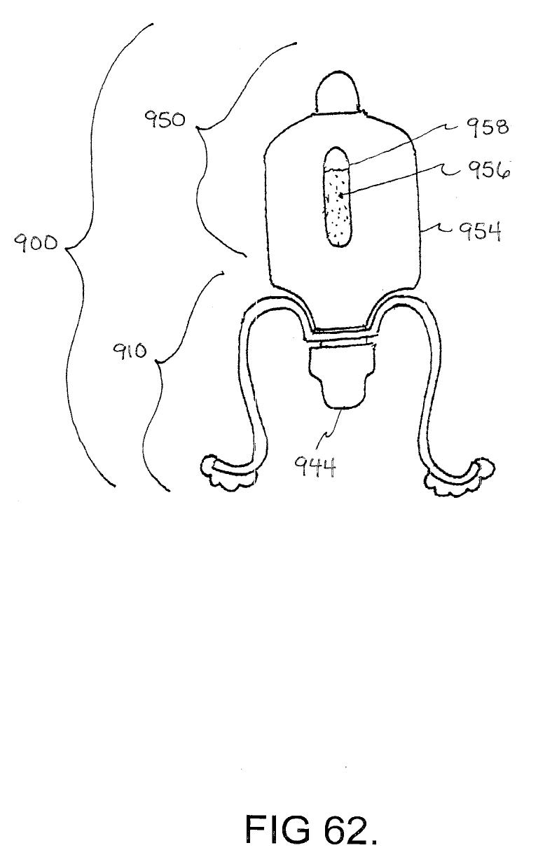

FIG. 62 is a front view of a sixth preferred embodiment of the invention 900. This embodiment includes a resilient leg portion 910 and a solution housing portion 950. A transparent window 958 located on the front panel 954 of the solution housing allows the user to see how much eyedrop solution 956 remains inside the housing 954. A removable and replaceable cap 944 encloses the dispensing tip 943 in an airtight manner.

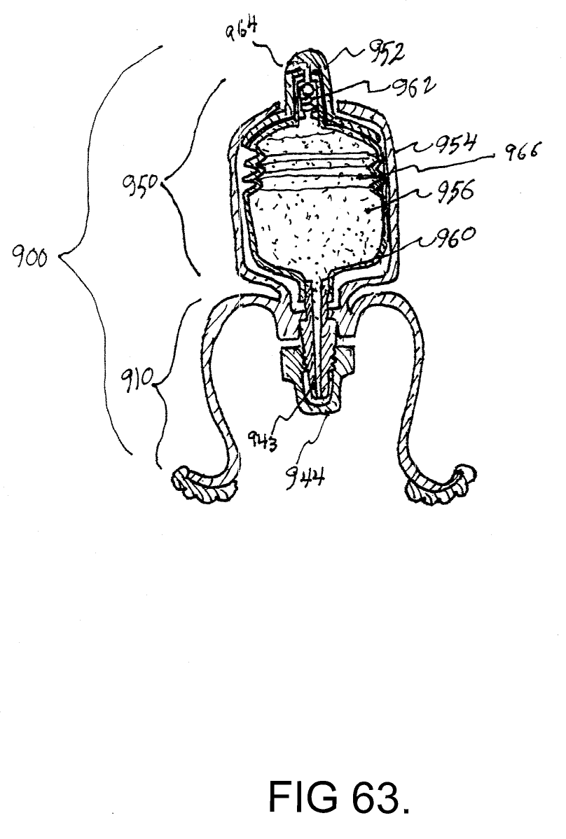

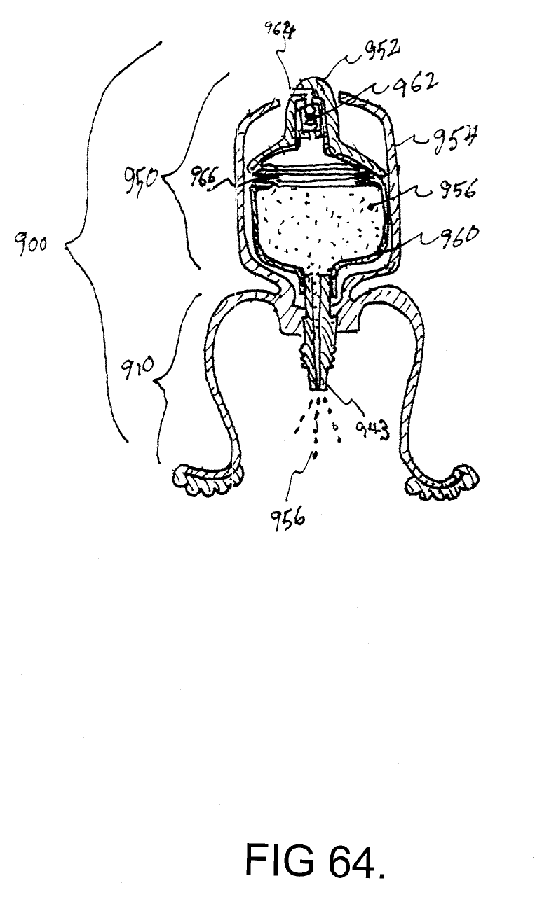

FIG. 63 shows a front section view of the fourth embodiment of the invention 900. A bladder 960 is located within hollow housing 954. The bladder 960 includes an accordion portion 966 that is compressed, as shown in FIG. 64, as the user presses down on the dispenser post 952. A one way valve 962 within the dispenser post 952 allows air to be drawn into valve orifice 964 as the bladder 960 returns to its original expanded shape as shown in FIG. 63. When the user presses down on dispenser post 952, a portioned amount of solution 956 within the bladder 960 is caused to be expelled from dispenser tip 943. Cap 944 is removably retained to the outer wall of dispenser tip 944 by standard means such as screw threads. This fourth embodiment design is easy and economical to manufacture resulting in a favorable final retail price to the consumer.

While the invention has been described in connection with a plurality of embodiments, it is not intended to limit the scope of the invention to the particular form set forth, but on the contrary, it is intended to cover such alternatives, modifications, and equivalents as may be included within the spirit and scope of the invention as defined by the appended claims. Furthermore, elements from one described embodiment can be included in the other described embodiments.

I claim:

1. A precision lid retracting eyedropper device comprising:
a hollow housing;
said housing having an aperture at its top most surface and a hollow cylindrical portion at its bottom most surface;
a pair of resilient downwardly facing legs integrally attached to the bottom surface of said hollow housing;
said legs terminating opposing J-shaped feet;
a plurality of friction inducing strips attached to the underside of said J-shaped feet;
said legs spaced sufficiently apart to allow a user to place one said J-shaped foot on the upper portion of the orbital ridge near the eyebrow and the other J-shaped foot on the lower portion of the orbital ridge near the cheek bone;
such that when the user squeezes said legs together, then rests said J-shaped feet on said upper and lower eye socket, and then releases said legs, the eyelids of the user are forced to remain open because of the outward spring action of said legs;
an eye drop solution storage chamber located within said hollow housing;
a pump assembly located within said hollow housing;
said pump assembly comprised of a resilient tubular member having an inwardly directed check valve frictionally inserted in one end and an outwardly directed check valve frictionally inserted at the opposite end;
an elongate piston member whose bottom end impinges on the central portion of said resilient tubular pump member and whose upper end exits said top housing aperture terminates at its top end in a push button;
said solution storage chamber connected to said inlet check valve by a first tubular member;
said outlet check valve of said pump assembly connected by a second tubular member to a downwardly disposed exit aperture; and
said second tubular member held in place by said cylindrical housing member;
so that when said user presses on said push button, said piston impinges on said resilient pump tube and forces a precise amount of eye drop solution out of said outwardly directed check valve and out said exit aperture and into said user's eye.

2. A precision lid retracting eyedropper device as claimed in claim 1 wherein said tubular pump assembly is oriented horizontally with respect to said piston allowing said piston to be at right angles to and to directly impinge upon on the central portion of said tubular member of said pump assembly.

3. A precision lid retracting eyedropper device as claimed in claim 1 wherein said solution storage chamber is constructed of resilient material allowing said chamber to collapse as solution is withdrawn from it so that no air can be found in the said solution chamber.

4. A precision lid retracting eyedropper device as claimed in claim 1 wherein said push button portion includes within it a standard counting mechanism and a window that displays advancing numbers, so that each time the said button is pushed, the said number counting mechanism advances by one digit

5. A precision lid retracting eyedropper device as claimed in claim 1 wherein said exit aperture consists of eighteen gauge or smaller tubing to decrease the chance of residual drops remaining on said aperture after each use.

6. The precision eye drop dispenser as claimed claim 1 wherein said pump assembly is positioned in a vertical position and said piston slidably impinges on a rigid elongate pivotal member terminating in a tube compressing foot member which can impinge upon said resilient tubular pump member causing said solution to be expelled into said user's eye.

7. The precision eye drop dispenser as claimed claim 1 wherein said push button is threadably engaged with said piston so that turning said push button clockwise creates a shorter stroke of said piston causing less eye drop solution to be expelled, and when said push button is turned counter clockwise, a longer stroke of said piston causes more eye drop solution to be expelled.

8. The precision eye drop dispenser as claimed claim 1 wherein said eye drop solution is stored in one or more separate single portion ampoules;
each said ampoule having an outlet port capped by a sterile cup shaped enclosure;
said ampoules being constructed of resilient material so that when said piston impinges upon the top of said ampoule, it squashes said ampoule, forcing said solution out and into said user's eye.

9. A precision lid retracting eyedropper device as claimed in claim 8 wherein said ampoules are removably attached to each other forming a string of ampoules which are then stored in a removably retainable cartridge that can attach to an ampoule compressing assembly.

10. The precision eye drop dispenser as claimed claim 1 further comprising a hollow, rigid, elongate cartridge body having a top cover and a bottom liquid dispensing tubular aperture;
said cartridge capable of retaining a liquid solution ;
a cartridge retaining housing having a hollow portion capable of removably retaining said cartridge;
said cartridge housing including an aperture to allow the said liquid dispensing tubular aperture to protrude out of the bottom of said housing;
said cartridge side wall having an aperture that is covered by a resilient membrane;
said cartridge including a inwardly biased check valve located in the top portion and an outwardly biased check valve mounted within said cartridge in front of said bottom dispensing aperture.
a pivotable spring biased L shaped arm located within said housing;
capable or impinging on said cartridge resilient membrane thereby forcing a precise amount of said liquid contained within said cartridge to be expelled from said dispensing aperture.

11. Precision lid retracting eyedropper device as claimed in claim 10 further comprising a pivotable spring biased activation arm;
said activation arm including a retaining tab at one end and an activation button at the opposite end;
said retaining tab capable of releasable engaging a mating tab located at the top end of said L shape so that said activation arm holds said L shaped arm in a ready position until said activation button is pushed by the user thereby causing said L shaped arm to be released causing the bottom portion of said L shaped arm to push into said cartridge membrane causing a precise amount of said liquid to be expelled from the said cartridge dispensing aperture;
said bottom portion of said L shaped arm including an adjustable threaded tip portion thereby allowing the user to adjust the distance between the said tip and the said membrane resulting adjusting the amount of solution dispensed per use

12. Precision lid retracting eyedropper device as claimed in claim 10 wherein said cartridge housing includes a hinged door that covers said hollow cartridge retaining portion;
said door held in a closed position by a standard latch assembly;
said door including an elongate aperture so that said user can view the amount of said liquid remaining in said cartridge.

13. Precision lid retracting eyedropper device as claimed in claim 10 wherein said cartridge and said cartridge housing are constructed of inexpensive injection molded plastic which allows for easy and inexpensive manufacturing.

14. Precision lid retracting eyedropper device as claimed in claim 10 wherein the tip of said L shaped arm includes a threaded head portion that can be turned clockwise or counterclockwise by the user to adjust the amount of drops that are released from said exit aperture during the solution dispensing operation.

15. Precision lid retracting eyedropper device as claimed in claim 1 wherein said downwardly facing resilient legs are hingedly attached to said housing so that said legs can each fold up one hundred and eighty degrees for compact storage and shipping.

16. Precision lid retracting eyedropper device as claimed in claim 1 wherein said exit aperture includes a standard spray inducing internal wall architecture that causes said eyedrop solution to impinge on the conjuntia of the user's eye in a fine spray format.

17. A preferred embodiment of the precision eye dropper comprised of a hollow housing;
a pair of downwardly facing, non-resilient legs terminating in a pair of concave shaped perpendicularly disposed feet and spaced so that one said foot engages the upper orbit socket of the user's eye and the other foot engages the lower orbit socket of the user's eye;
said housing containing a first flexible bladder,
a second flexible bladder, a first and a second one way valve,
a rubber stopper insert, a plunger rod and a dispensing tip;
said first bladder being doughnut shaped and having an integral tube extension that attaches to said first check valve;
said plunger rod extending down through said doughnut shaped first bladder;
said first check valve communicating an orifice at the top of said second bladder;
said second check valve communicating with an orifice at the bottom of said second bladder;
the bottom of said second check valve communicating with said dispensing tip;
so that when a person presses on the top of said plunger rod, liquid stored within said first bladder is forced through said extension tube into said first one way valve and into said second bladder;
said plunger causing compression in said second bladder and causing said eye drop solution stored within said second bladder to be expelled through said second one way valve and out of said dispensing tip into the user's eye.

18. A precision eye drop dispenser as claimed in claim 17 wherein a sterile syringe filled with eye drop solution is packaged in a sterile container;
the needle tip of said syringe being closed off and said barrel of said needle having a side aperture so that when said user injects said needle into said rubber stopper, said eye drop solution is injected into said bladder in a sterile fashion and where no loose rubber particulates can enter said first bladder; said aperture created by said needle in said rubber stopper closes up in an airtight fashion when said needle is removed from said rubber stopper.

19. A precision eye drop dispenser as claimed in claim 17 wherein the bottom of said feet are over-molded with a rubber-like compound and are ribbed for non-slip effect.

20. A precision eye drop dispenser as claimed in claim 17 wherein the junction between said leg and said foot is thin so that said foot can flex lightly to accommodate the variations of said user's orbital socket.

21. A precision eye drip dispenser as claimed in claim 17 wherein said dispenser is enclosed packaged in an air tight bag along with a syringe filled with eye drop solution and ready to be injected into said dispenser in a sterile fashion.

22. A precision eye drop dispenser comprising; a hollow housing; a pair of non-resilient downwardly disposed legs terminating in a pair of concave feet;
said legs parallel spaced and fixedly attached to the underside of said hollow housing;
said hollow housing enclosing a U shaped track, a drive disk, a ratchet drive disk, a spring biased plunger rod, a ratchet drive arm and a pawl for said ratchet drive arm;
said drive disk and said ratchet disk concentrically and fixedly attached to each other;
said drive disk having a shaft member;
said shaft member rotatably retained within a centrally located aperture on a flat wall of said housing;
said ratchet drive arm attached to said plunger rod in a spaced and parallel manner;
said ratchet drive arm terminating at its lowest point in a pawl assembly; said pawl capable of forcefully and upwardly engaging said ratchet drive disk thereby causing a plurality of radially displaced posts attached to the perimeter of said drive disk to rotate in a clockwise manner;
and in turn causing a portion of an eye drop portion retaining strip, slidably retained within said track, to be advanced forward one portion position;
said plunger rod slidably retained in a vertical manner within said hollow housing;
said plunger rod tip capable of depressing a dome shaped portion located on the top side of said eye drop retaining strip thereby causing a precise portion of said eye drop solution to be dispensed out of a cone shaped, cap covered portion affixed to the bottom side of said eye drop portion retaining strip.

23. The precision eye drop dispenser as claimed claim 1 wherein that substitutes resilient legs as used in the preferred embodiment instead of said non-resilient legs.

24. The precision eye drop dispenser as claimed claim 1 wherein includes a motorized cam mechanism that automatically drives said dispensing plunger downward when the user presses a dispensing button.

25. The precision eye drop dispenser as claimed claim 1 wherein said push button portion encases a check valve which communicates with a solution containing bladder stored within a hollow housing; said bladder walls having an accordion portion allowing said bladder to be compressed when a user presses down on said push button causing said solution to be expelled in a precise portioned manner from a dispenser tip located at the bottom of said housing and where air is drawn into said bladder from said check valve as said bladder returns to its expanded position.
